(19) United States
(12) Patent Application Publication
(10) Pub. No.: US 2018/0135019 A1
(43) Pub. Date: May 17, 2018

(54) METHODS AND PRODUCTS FOR ENRICHING AND ISOLATING STEM CELLS

(71) Applicant: ADELAIDE RESEARCH & INNOVATION PTY LTD, Adelaide (AU)

(72) Inventors: Andrew Zannettino, Highbury (AU); Stan Gronthos, Colonel Light Gardens (AU); Stephen Fitter, Blair Athol (AU)

(21) Appl. No.: 15/578,139

(22) PCT Filed: Jun. 1, 2016
(86) PCT No.: PCT/AU2016/050438

§ 371 Date: Nov. 29, 2017

(30) Foreign Application Priority Data
Jun. 1, 2015 (AU) 2015902032

(51) Int. Cl. C12N 5/0775; G01N 33/50

(52) U.S. Cl. CPC C12N 5/0663; C12N 2501/07; G01N 33/5073

(57) ABSTRACT

The present disclosure relates to methods and products for enriching and isolating stromal stem cells. Certain embodiments of the present disclosure provide a method of enriching for immature, uncommitted stromal stem cells from a population of cells comprising stromal stem cells, the method comprising enriching stromal stem cells from the population of cells using a HSC70 binding agent.

### PRIORITY CLAIM

This application claims priority to Australian provisional patent application number 2015902032 filed on 1 Jun. 2015, the content of which is hereby incorporated by reference.

### FIELD

The present disclosure relates to methods and products for enriching and isolating stromal stem cells. The present disclosure also relates to enriched and isolated stromal stem cells.

### BACKGROUND

The clinical use of stromal stem cells represents an emerging avenue for the treatment of a number of pathologic conditions. A variety of different types of stromal stem cells exist and many of these have potential for use in stem cell based therapies.

However the full clinical potential of stromal stem cells has been hindered, in large part by a lack of uniformity in the methods used for cell isolation, culture and characterization, and the fact that that the selection techniques produce heterogeneous populations of cells.

Mesenchymal stem cells (MSCs) are one type of stromal stem cell which have significant clinical potential. MSCs used clinically are isolated based on limited criteria that include the ability to adhere to tissue culture plastic, the presence and absence of a limited number of cell surface antigens and to have tri-lineage differentiation potential. While these plastic adherent (PA-MSC) cultures have shown some clinical efficacy, the PA-MSC cell population is very heterogeneous and only contains a small number of immature cells which are more efficacious. For this reason the clinical utility of PA-MSC has been limited.

Immunoselection of some stromal stem cells has also been utilised. For example, the monoclonal antibody STRO-1 is a tool for prospective immunoselection of MSC precursors, as this monoclonal antibody binds an antigen expressed on the surface of a small population (˜10%) of adult bone marrow mononuclear cells (BMMNCs), a proportion of which are clonogenic.

However, it has also become apparent that the population of cells selected using STRO-1 is highly heterogeneous. In addition, the STRO-1 antibody is denatured in the viral inactivation step required for GMP compliance and therefore cannot readily be used for the isolation of MSCs for clinical applications.

For these reasons there remains a need to identify new markers that will result in more therapeutically potent stromal stem cell populations than are currently possible.

### SUMMARY

The present disclosure relates to methods and products for enriching and isolating stromal stem cells.

Certain embodiments of the present disclosure provide a method of enriching for immature, uncommitted stromal stem cells from a population of cells comprising stromal stem cells, the method comprising enriching stromal stem cells from the population of cells using a HSC70 binding agent.

Certain embodiments of the present disclosure provide a method of enriching for immature, uncommitted stromal stem cells from a population of cells comprising stromal stem cells, the method comprising enriching stromal cells from the population of cells expressing HSC70 as a cell surface marker and thereby enriching for immature, uncommitted stromal stem cells.

Certain embodiments of the present disclosure provide a method of enriching for immature, uncommitted stromal stem cells, the method comprising:
providing a population of cells comprising stromal stem cells;
exposing the population of cells to a HSC70 binding agent; and
enriching stromal cells from the population of cells using the HSC70 binding agent bound to cells.

Certain embodiments of the present disclosure provide a method of enriching for immature, uncommitted stromal stem cells from a population of cells comprising stromal stem cells, the method comprising enriching stromal cells from the population of cells by detecting cells that express HSC70 as a cell surface marker.

Certain embodiments of the present disclosure provide a method of enriching for immature, uncommitted mesenchymal stem cells from a population of cells comprising stromal stem cells, the method comprising enriching mesenchymal stem cells from the population of cells using a HSC70 binding agent.

Certain embodiments of the present disclosure provide a method of enriching for immature, uncommitted mesenchymal stem cells from a population of cells comprising stromal stem cells, the method comprising enriching mesenchymal stem cells from the population of cells expressing HSC70 as a cell surface marker and thereby enriching for immature, uncommitted stromal stem cells.

Certain embodiments of the present disclosure provide use of HSC70 as a marker for enriching, isolating and/or identifying immature, uncommitted stromal stem cells.

Certain embodiments of the present disclosure provide use of a HSC70 binding agent for enriching, isolating and/or identifying immature, uncommitted stromal stem cells.

Certain embodiments of the present disclosure provide a method of isolating immature, uncommitted stromal stem cells from a population of cells comprising stromal stem cells, the method comprising isolating stromal stem cells from the population of cells using a HSC70 binding agent.

Certain embodiments of the present disclosure provide a method of isolating immature, uncommitted stromal stem cells, the method comprising:
providing a population of cells comprising stromal stem cells;
exposing the population of cells to a HSC70 binding agent; and
isolating stromal cells from the population of cells using the HSC70 binding agent bound to cells.

Certain embodiments of the present disclosure provide a method of isolating immature, uncommitted stromal stem cells from a population of cells comprising stromal stem cells, the method comprising isolating stromal stem cells expressing HSC70 from the population of cells.

Certain embodiments of the present disclosure provide a method of isolating immature, uncommitted mesenchymal stem cells from a population of cells comprising stromal stem cells, the method comprising isolating mesenchymal stem cells from the population of cells using a HSC70 binding agent.

Certain embodiments of the present disclosure provide a method of isolating immature, uncommitted mesenchymal stem cells from a population of cells comprising stromal stem cells, the method comprising isolating mesenchymal stem cells expressing HSC70 from the population of cells.

Certain embodiments of the present disclosure provide one or more isolated stromal stem cells, the stromal stem cells expressing HSC70 as a cell surface marker and not substantially expressing HSP70 as a cell surface marker.

Certain embodiments of the present disclosure provide one or more isolated stromal stem cells, the stromal stem cells comprising HSC70⁺ and HSP70⁻ cell surface markers.

Certain embodiments of the present disclosure provide one or more isolated stromal stem cells, the stromal stem cells comprising HSC70^{bright} and HSP70^{dim} cell surface markers.

Certain embodiments of the present disclosure provide one or more isolated stromal stem cells, the stromal stem cells expressing a STRO-1 antigen as a cell surface marker and not substantially expressing HSP70 as a cell surface marker.

Certain embodiments of the present disclosure provide one or more isolated stromal stem cells, the stromal stem cells comprising STRO-1⁺ and HSP70⁻ cell surface markers.

Certain embodiments of the present disclosure provide one or more isolated stromal stem cells, the stromal stem cells comprising STRO-1^{bright} and HSP70^{dim} cell surface markers.

Certain embodiments of the present disclosure provide a method of enriching for immature, uncommitted stromal stem cells from a population of cells comprising stromal stem cells, the method comprising enriching for stromal stem cells expressing a STRO-1 antigen as a cell surface marker and not substantially expressing HSP70 as a cell surface marker from the population of cells.

Certain embodiments of the present disclosure provide a method of enriching for stromal stem cells from a population of cells comprising stromal stem cells, the method comprising enriching for STRO-1⁺ HSP70⁻ stromal stem cells from the population of cells.

Certain embodiments of the present disclosure provide a method of isolating immature, uncommitted stromal stem cells from a population of cells comprising stromal stem cells, the method comprising isolating stromal stem cells expressing a STRO-1 antigen as a cell surface marker and not substantially expressing HSP70 as a cell surface marker from the population of cells.

Certain embodiments of the present disclosure provide a method of isolating immature, uncommitted stromal stem cells from a population of cells comprising stroma stem cells, the method comprising isolating STRO-1⁺ HSP70⁻ stromal cells from the population of cells.

Certain embodiments of the present disclosure provide a method of identifying an immature, uncommitted stromal stem cell, the method comprising identifying a stromal stem cell that expresses a HSC70 cell surface marker.

Certain embodiments of the present disclosure provide a method of identifying an agent for enriching, isolating and/or identifying an immature, uncommitted stromal stem cell, the method comprising:
determining whether a candidate agent binds to HSC70 on a stromal stem cell; and
identifying the candidate agent as an agent for enriching, isolating and/or
identifying an immature, uncommitted stromal stem cells.

Certain embodiments of the present disclosure provide a kit for enriching and/or isolating immature, uncommitted stromal stem cells, the kit comprising a HSC70 binding agent.

Certain embodiments of the present disclosure provide a method of identifying a HSC70 binding agent, the method comprising identifying an agent that binds to HSC70 but does not substantially bind to HSP70.

Certain embodiments of the present disclosure provide an isolated and/or a non-naturally occurring polypeptide comprising the amino acid sequence according to SEQ ID NO.4 and/or a variant thereof.

Certain embodiments of the present disclosure provide a STRO-1 antagonist comprising an amino acid sequence according to SEQ ID NO. 4 and/or a functional variant thereof.

Certain embodiments of the present disclosure provide a method of inhibiting STRO-1 binding to a cell, the method comprising using an agent comprising an amino acid sequence according to SEQ ID NO.4 and/or a functional variant thereof to reduce the binding of STRO-1 to the cell.

Other embodiments are disclosed herein.

### BRIEF DESCRIPTION OF THE FIGURES

Certain embodiments are illustrated by the following figures. It is to be understood that the following description is for the purpose of describing particular embodiments only and is not intended to be limiting with respect to the description.

FIG. 1 shows that the STRO-1 antigen is localised to cholesterol rich micro-domains.

FIG. 2 shows that STRO-1 binds to a 70 kDa protein

FIG. 3 shows that STRO-1 binds to heat shock cognate 70 (HSC70; HSPA8)

FIG. 4 shows that STRO-1 binds to HSC70 and HSP70 and cell surface binding of STRO-1 does not correlate with HSC70 or HSP70 protein expression.

FIG. 5 shows that HSC70 is present on the surface of UE7T-13 cells and pre-incubation of STRO-1 with recombinant HSC70 blocks STRO-1 binding to UE7T-13 cells.

FIG. 6 shows that STRO-1 binds to the ATPase domain of HSC70.

FIG. 7 shows fine mapping the STRO-1 epitope on HSC70.

FIG. 8 shows that recombinant HSC70 blocks STRO-1 binding to the cell surface.

FIG. 9 shows flow cytometry histograms of the fluorescence signal generated following the incubation of UE7T-13 cells with an anti-Salmonella IgM negative control, 1A6.12 (A), STRO-1 pre-incubated with GST (B), STRO-1 pre-incubated with GST-L393 (C). An overlay of A-C(D). Bound IgM anti-body was detected using anti-IgM-PE.

FIG. 10 shows STRO-1 +ve/HSP70 −ve fraction of human BMMNCs contains all CFU-F activity.

FIG. 11 shows 20-202s and STRO-1 have different antigenic specificities.

### DETAILED DESCRIPTION

The present disclosure relates to the enrichment and/or isolation of precursor stromal stem cells.

The present disclosure is based, at least in part, upon the recognition that heat shock cognate 70 (HSC70) protein is a cell surface marker of mesenchymal stem cells (MSC) that have an immature and an uncommitted phenotype. The recognition that HSC70 is expressed on the cell surface of only this precursor stem cell population provides a unique identifier for prospective enrichment/isolation of immature, uncommitted stromal stem cells generally, and particularly for research and/or medical applications.

The identification of HSC70 as a cell surface antigen associated with an immature phenotype of stromal stem cells is also unexpected as HSC70 is an intracellular heat shock cognate protein and the protein is not considered typically to be a cell surface marker and has no transmembrane domain.

Certain embodiments of the present disclosure are directed to methods and products that have one or more combinations of advantages. For example, some of the advantages of the embodiments disclosed herein include one or more of the following: providing a new cell surface marker for the enrichment and/or isolation of stromal stem cells; identification of a stromal stem cell surface marker associated with an immature stem cell-like phenotype; providing binding agents that target new stromal stem cell surface markers; providing new methods for prospectively enriching and/or isolating stromal stem cells, for example for possible clinical use; providing new methods of enriching and/or isolating stromal stem cells which are not based on criteria such as ability to adhere to tissue culture plastic, the presence and absence of previous cell surface antigens, and/or certain differentiation potential; improving aspects of previous methods which typically produce populations that are heterogeneous; providing new methods to isolate more uniform and/or functionally superior populations of stromal stem cells; determination of the cell surface antigens recognised by STRO-1; improving the characteristics of stromal stem cells isolated utilising STRO-1; to provide new methods and products with one or more advantages, and/or to provide a commercially useful choice. Other advantages of certain embodiments of the present disclosure are also disclosed herein.

Certain embodiments of the present disclosure provide use of HSC70 as a marker for enriching, isolating and/or identifying immature, uncommitted stromal stem cells.

Certain embodiments of the present disclosure provide use of a HSC70 binding agent for enriching, isolating and/or identifying immature, uncommitted stromal stem cells.

Certain embodiments of the present disclosure provide a method of enriching for immature, uncommitted stromal stem cells by enriching for stromal stem cells having surface expression of HSC70.

Certain embodiments of the present disclosure provide a method of enriching for immature, uncommitted stromal stem cells using a HSC70 binding agent.

Certain embodiments of the present disclosure provide a method of enriching for immature, uncommitted stromal stem cells from a population of cells comprising stromal stem cells, the method comprising enriching stromal stem cells from the population of cells using a HSC70 binding agent.

Certain embodiments of the present disclosure provide a method of enriching for immature, uncommitted stromal stem cells from a population of cells comprising stromal stem cells, the method comprising enriching stromal stem cells from the population of cells using a HSC70 binding agent to enrich for immature, uncommitted stromal stem cells.

Certain embodiments of the present disclosure provide a method of enriching for immature, uncommitted stromal stem cells from a population of cells comprising stromal stem cells, the method comprising enriching stromal stem cells having surface expression of HSC70.

The term “stromal stem cells” as used herein refers to a self-renewing multipotent population of cells present in any organ that can give rise to the connective tissue elements of the tissue of origin and potentially other tissues. This includes, for example, cells that have the ability under some conditions to differentiate into cells of mesodermal lineage such as osteoblasts, myocytes, adipocytes, myelosupportive stroma and under some conditions to differentiate into non-mesodermal cells, such as neuron-like cells.

As described herein, HSC70 has been identified as a marker for the purification of immature, uncommitted stromal stem cells. The cells so isolated are precursors to immature and mature stromal cells including, but not limited to, mesenchymal stromal cells, mesenchymal stem cells and skeletal stem/progenitor cells.

Stromal stem cells may be identified by a method known in the art, for example as described in Gronthos S. et al. (2003) J. Cell Sci. 116 (Pt 9):1827-35.

For example, markers for stromal stem cells typically include positive markers such as Leptin-Receptor, Low affinity NGF-receptor (CD271), EGF-receptor, PDGF-receptor (CD140a,b), IGF-1-receptor, FGF-1,2,3,4-receptor, BMP-receptor, TGF beta-receptor, Alkaline phosphatase, Thrombomodulin, Vimentin, Integrin beta 5, Nestin, Stem cell factor, Collagen type I, Collagen type VI, HSP90, RANKL, STRO-1 antigen, CXCL12, CD10, CD13, CD29, CD44, CD49a,b,d,e,f, CD51, CD54, CD58, CD61, CD73CD90, CD105, CD144, CD146, CD166, CD184, and CD200, and/or the absence of markers such as c-fms Glycophorin-A, HLA-DR, Von Willebrand Factor, E-Selectin, CD3, CD4, CD11b, CD14, CD18, CD19, CD20, CD31, CD33, CD34, CD38, CD40, CD44, CD45, CD80, CD86, and CD117 (c-kit).

In certain embodiments the stromal stem cells comprise mesenchymal stem cells. In certain embodiments, the stromal stem cells comprise osteochondral stem cells. Methods for identifying mesenchymal cells and osteochondral cells are known in the art. Other types of stem cells are contemplated.

In certain embodiments, the population of cells comprises stromal stem cells obtained, and/or derived or arising from, placenta, umbilical cord, umbilical cord blood, tooth bud tissue, dentine/pulp tissue, periodontal ligament, gingival, skin, hair, follicle, amniotic fluid, adipose tissue, smooth muscle, skeletal muscle, tendon, ligament, bone, cartilage, bone marrow and/or peripheral blood. Other types of sources are contemplated. Methods for producing such cells are known in the art, for example as described in Gronthos S., Zannettino A C W, Kortesidis A, Shi S, Graves S E, Hay S J, Simmons P J (2003) Molecular and cellular characterisation of highly purified human bone marrow stromal stem cells. Journal of Cell Science 116: 1827-1835; Shi S. and Gronthos S. (2003) Perivascular Niche of Postnatal Mesenchymal Stem Cells in Human Bone Marrow and Dental Pulp. Journal of Bone and Mineral Research 18(4): 696-704; Zannettino A C W, Paton S, Kortesidis A, Khor F, Itescu S, Gronthos S (2007) Human Mulipotential Stromal Stem Cells are Derived from a Discrete Subpopulation of STRO-1 bright/CD34-/CD45-/Glycophorin-A-Bone Marrow Cells. Haematologica 92 1707-1708; and Zannettino A C, Paton S, Arthur A, Khor F, Itescu S, Gimble J M, Gronthos S (2008) Multipotential human adipose-derived stromal stem cells exhibit a perivascular phenotype in vitro and in vivo. Journal of Cellular Physiology 214(2):413-421.

In certain embodiments, the population of cells comprises stromal stem cells arising from pluripotent stem cells, induced pluripotent stem cells, cells arising from somatic nuclear transfer, and/or adult stem cells. Methods for producing such cells are known in the art.

The term “enriching” and related terms such as “enrich” and “enriched” as used herein includes increasing the proportion of one or more particular species, such as particular cells type, in a mixture of species (such as other cells). Enrichment may include one or more steps of enriching.

In certain embodiments, the enriching of immature, uncommitted stromal stems cells comprises an enrichment of at least 2 fold, at least 3 fold, at least 4 fold, at least 5 fold, at least 6 fold, at least 7 fold, at least 8 fold, at least 9 fold, at least 10 fold, at least 20 fold, at least 50 fold, at least 100 fold, at least 1000 fold, at least 10,000 fold or at least 50,000 fold. Other levels of enrichment are contemplated.

In certain embodiments, the enriching results in a population of cells whereby the immature, uncommitted stromal stem cells comprises at least 1%, at least 2%, at least 5%, at least 10%, at least 20%, at least 30%, at least 40%, at least 50%, at least 60%, at least 70%, at least 80%, at least 90%, at least 95% or at least 99% of the total cells in the population. Other levels of enrichment are contemplated. In certain embodiments, the enriching results in a population of cells whereby the immature, uncommitted stromal stem cells comprise about 50% or more of the total cells in the population. In certain embodiments, the enriching results in a population of cells whereby the immature, uncommitted stromal stem cells comprise about 900/% or more of the total cells in the population. In certain embodiments, the enriching results in a population of cells whereby the immature, uncommitted stromal stem cells comprise about 95% or more of the total cells in the population. In certain embodiments, the enriching results in a population of cells whereby the immature, uncommitted stromal stem cells comprise about 99% or more of the total cells in the population.

In certain embodiments, the enriching comprises enriching stromal stem cells from a population of cells so that the enriched immature, uncommitted stromal stem cells comprise substantially the only cells present.

HSC70 is a member of the HSP70 family of proteins, which contains both heat-inducible and constitutively expressed members. HSC70 is encoded by the HSPA8 gene. Of its functions, HSC70 appears to function as a chaperone, and binds to nascent polypeptides to facilitate correct folding. Alternatively spliced transcript variants encoding different isoforms have been found for the HSPA8 gene.

In the human, the protein sequence has the UniProt identifier P11142-HSP7C_HUMAN.

The amino acid sequence of the main human isoform is as follows (SEQ ID NO.1):

(SEQ ID NO. 1)
MSKGPAVGID LGTTYSCVGV FQHGKVEIIA NDQGNRTTPS
YVAFTDTERL IGDAAKNQVA MNPTNTVFDA KRLIGRRFDD
AVVQSDMKHW PFMVVNDAGR PKVQVEYKGE TKSFYPEEVS
SMVLTKMKEI AEAYLGKTVT NAVVTVPAYF NDSQRQATKD
AGTIAGLNVL RIINEPTAAA IAYGLDKKVG AERNVLDFDL
GGGTFDVSIL TIEDGIFEVK STAGDTHLGG EDFDNRMVNH
FIAEFKRKHK KDISENKRAV RRLRTACERA KRTLSSSTQA
SIEIDSLYEG IDFYTSITRA RFEELNADLF RGTLDPVEKA
LRDAKLDKSQ IHDIVLVGGS TRIPKIQKLL QDFFNGKELN
KSINPDEAVA YGAAVQAAIL SGDKSENVQD LLLLDVTPLS
LGIETAGGVM TVLIKRNTTI PTKQTQTFTT YSDNQPGVLI
QVYEGERAMT KDNNLLGKFE LTGIPPAPRG VPQIEVTFDI
DANGILNVSA VDKSTGKENK ITITNDKGRL SKEDIERMVQ
EAEKYKAEDE KQRDKVSSKN SLESYAFNMK ATVEDEKLQG
KINDEDKQKI LDKCNEIINW LDKNQTAEKE EFEHQQKELE
KVCNPIITKL YQSAGGMPGG MPGGFPGGGA PPSGGASSGP
TIEEVD

The nucleotide sequence of the human gene is as provided in NCBI Reference Sequence: NG_029473.1.

HSC70, its homologues, paralogues and/or variants thereof, and HSC70 in other species, may all be readily identified and are included within the scope of the present disclosure. Methods for determining related genes and proteins, including related genes and proteins in other species are known, and include for example nucleic acid and protein alignment programs, such as the BLAST suite of alignment programs.

In certain embodiments, the method of enriching uses a human HSC70 and/or the method of enriching is used to enrich cells from a human subject.

In certain embodiments, the method of enriching uses a non-human HSC70 and/or the method of enriching is used to enrich cells from a non-human subject. For example, the method may be used to enrich immature, uncommitted stromal stem cells from a mammalian subject, a livestock animal (such as a horse, a cow, a sheep, a goat, a pig), a domestic animal (such as a dog or a cat) and other types of animals such as, non-human primates, rabbits, rats, mice and laboratory animals. Veterinary applications of the present disclosure are contemplated.

Methods for detecting expression of HSC70 are known in the art. Methods for using markers for enriching cells are known in the art, such as flow cytometry or affinity purification methods.

In certain embodiment, a HSC70 binding agent is used to enrich immature, uncommitted stromal stem cells.

In certain embodiment, a HSC70 binding agent is used to enrich immature, uncommitted stromal stem cells from a human subject.

In certain embodiments, a HSC70 binding agent is used to enrich immature, uncommitted stromal stem cells from a non-human subject. Non-human subjects are as described herein.

In certain embodiments, the HSC70 binding agent comprises one or more of an antibody and/or an antigen binding part thereof, a small molecule, a nucleic acid, an aptamer, a polypeptide, a protein, ligand or a ligand mimetic. Other types of agents are contemplated.

In certain embodiments, the method comprises enriching for cells that express HSC70 as a cell surface marker.

In certain embodiments, the method comprises binding of a HSC70 binding agent to cell surface HSC70. In certain embodiments, the HSC70 binding agent binds to cell surface HSC70. In certain embodiments, the method comprises binding of the HSC70 binding agent to cell membrane associated HSC70. Methods for detecting the binding of agents to cell surface markers are known in the art.

In certain embodiments, the HSC70 binding agent comprises a ligand to the HSC70 protein. In certain embodiments, the ligand comprises all or part of a ligand to HSC70. In certain embodiments, the ligand comprises all or part of a naturally-occurring ligand to HSC70. In certain embodiments, the HSC70 binding agent comprises a soluble portion of a ligand to a HSC70 ligand.

In certain embodiments, the HSC70 binding agent binds to HSC70 but does not substantially bind to HSP70. In certain embodiments, the HSC70 binding agent comprises a disassociation constant (Kd) for binding to HSC70 that is at least 10 fold, at least 100 fold, or at least 1000 fold greater than the Kd of binding to HSP70. Methods for determining affinity of binding are known in the art.

In certain embodiments, the method comprises using a HSC70 binding agent bound to cells to enrich cells.

Certain embodiments of the present disclosure provide a method of enriching for immature, uncommitted stromal stem cells from a population of cells comprising stromal stem cells, the method comprising enriching stromal stem cells from the population of cells using a HSC70 binding agent bound to cells.

In certain embodiments, the HSC70 binding agent comprises all or part of a EWI-2 protein (CD316) and/or a variant thereof. EWI-2 is a HSC70 ligand, as described in Kettner et al. (2007) Mol Cell Biol. 27(21):7718-26.

In certain embodiments, the HSC70 binding agent comprises all or part of a EWI-2 protein (CD316) and/or a variant thereof. In certain embodiments, the HSC70 binding agent comprises a soluble form of a EWI-2 protein (CD316) and/or a variant thereof. In certain embodiments, the HSC70 binding agent comprises all or part of an extracellular domain/region of a EWI-2 protein (CD316) and/or a variant thereof, for example a sEWI-2-hIg protein with four extracellular domains, including amino acids 1 to 574 fused to the J-CH₂—CH₃ domains of the hIgG heavy chain, as described in Kettner et al. (2007) Mol Cell Biol. 27(21):7718-26.

In certain embodiments, the HSC70 binding agent comprises an antibody and/or an antigen binding part thereof.

In certain embodiments, the HSC70 binding agent comprises an antibody, and/or antigen binding part thereof, to a HSC70 protein. Antibodies to HSC70 protein may be produced or obtained commercially, such as the promiscuous STRO-1 antibody as described herein. In certain embodiments, the HSC70 binding agent comprises an antibody to human HSC70.

Polyclonal and monoclonal anti-HSC70 antibodies may be produced or obtained commercially, for example from sources such as Abcam and ThermoFisher Scientific.

In certain embodiments, the HSC70 binding agent comprises an antibody, and/or an antigen binding part thereof, that binds to HSC70 but does not substantially bind to HSP70. In certain embodiments, the anti-HSC70 antibody comprises a dissociation constant Kd for binding to HSC70 that is at least 10 fold, at least 100 fold, or at least 1000 fold greater than the Kd of binding to HSP70. Methods for determining affinity of binding are known in the art. In certain embodiments, the anti-HSC70 antibody does not bind substantially to HSP70.

Monoclonal antibodies, and/or an antigen binding part thereof, that bind to an epitope in HSC70 may be produced by a method known in the art.

In certain embodiments, an antibody and/or an antigen binding part thereof binds to one or more epitopes in HSC70 but does not substantially bind to HSP70.

The term “antibody” as used herein refers to an immunoglobulin molecule with the ability to bind an antigenic region of another molecule, and includes monoclonal antibodies, polyclonal antibodies, multivalent antibodies, chimeric antibodies, multispecific antibodies, diabodies and fragments/parts of an immunoglobulin molecule or combinations thereof that have the ability to bind to the antigenic region of another molecule with the desired affinity including a Fab, Fab′, F(ab′)₂, Fv, a single-chain antibody (scFv) or a polypeptide that contains at least a portion of an immunoglobulin (or a variant of an immunoglobulin) that is sufficient to confer specific antigen binding, such as a molecule including one or more Complementarity Determining Regions (CDRs).

In this regard, an immunoglobulin is a tetrameric molecule, each tetramer being composed of two identical pairs of polypeptide chains, each pair having one light chain and one heavy chain. The amino-terminal portion of each chain includes a variable region of about 100 to 110 or more amino acids that is primarily responsible for antigen recognition. The carboxy-terminal portion of each chain defines a constant region primarily responsible for effector function. Human light chains are classified as K and λ light chains. Heavy chains are classified as μ, Δ, γ, α, or ε and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. The variable regions of each light/heavy chain pair form the antibody binding site, with the result that an intact immunoglobulin has two binding sites. The variable regions further include hypervariable regions that are directly involved in formation of the antigen binding site. These hypervariable regions are usually referred to as Complementarity Determining Regions (CDR). The intervening segments are referred to as Framework Regions (FR). In both light and heavy chains there are three CDRs (CDR-I to CDR-3) and four FRs (FR-I to FR-4).

In certain embodiments, the antigen-binding fragment/part comprises a Fab, Fab′, F(ab′)₂, Fd, Fv, a single-chain antibody (scFv), a chimeric antibody, a diabody or a polypeptide that contains at least a portion of an immunoglobulin that is sufficient to confer specific antigen binding.

A Fab fragment is a monovalent fragment consisting of the VL, VH, CL and CH I domains. A F(ab′)₂ fragment is a bivalent fragment including two Fab fragments linked by a disulphide bridge at the hinge region. A Fd fragment consists of the VH and CH I domains. A Fv fragment consists of the VL and VH domains of a single arm of an antibody. A dAb consists of a VH domain. A single chain antibody (scFv) is an antibody in which VL and VH regions are paired to form a monovalent molecule via a synthetic linker that enable them to be made as a single protein chain. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites.

Antibody fragments, or parts of an antibody, that contain specific binding sites may be generated by a known method. Methods for producing antigen-binding fragments or portions/parts of antibodies are known in the art, for example as described in “Antibody Engineering: Methods and Protocols” (2004) ed. by B. K. C. Lo Humana Press, herein incorporated by reference; and “Antibody Engineering: A Practical Approach” (1996) ed. by J. McCafferty, H. R. Hoogenboom and D J. Chriswell Oxford University Press, herein incorporated by reference. For example, F(ab′)₂ fragments can be produced by pepsin digestion of the antibody molecule, and Fab fragments can be generated by reducing the disulfide bridges of the F(ab′)₂ fragments. Alternatively, Fab expression libraries may be constructed to allow rapid and easy identification of monoclonal Fab fragments with the desired specificity, as described for example in Huse, W. D. et al. (1989) Science 254: 1275-1281, herein incorporated by reference.

Antibodies may be generated using known methods. For the production of antibodies, various hosts including goats, rabbits, rats, mice, humans, and others, may be immunized by injection with an appropriate antigen. Depending on the host species, various adjuvants may be used to increase an immunological response. Such adjuvants include Freund's adjuvant, mineral gels such as aluminium hydroxide, and surface-active substances such as lysolecithin, pluronic polyols, polyanions, peptides, oil emulsions, keyhole limpet hemocyanin, and dinitrophenol. Adjuvants are commercially available.

In certain embodiments, the antibody is a polyclonal antibody. A polyclonal antibody is a mixture of antibodies having different antigen specificities. Methods for producing and isolating polyclonal antibodies are known in the art. In general, polyclonal antibodies are produced from B-lymphocytes. Typically polyclonal antibodies are obtained directly from an immunized subject, such as an immunized animal.

In certain embodiments, the antibody is a monoclonal antibody. Monoclonal antibodies may be prepared using a technique that provides for the production of antibody molecules by continuous isolated cells in culture. These include, but are not limited to, the hybridoma technique, the human B-cell hybridoma technique, and the EBV-hybridoma technique. Methods for the preparation of monoclonal antibodies include for example Kohler et al. (1975) Nature 256:495-497, herein incorporated by reference; Kozbor et al. (1985) J. Immunol. Methods 81:31-42, herein incorporated by reference; Cote et al. (1983) Proc. Natl. Acad. Sci. 80:2026-2030, herein incorporated by reference; and Cole et al. (1984) Mol. Cell Biol. 62: 109-120, herein incorporated by reference.

In certain embodiments, the antibody and/or an antigen binding fragment thereof comprises an isolated antibody. Methods for producing and isolating polyclonal and monoclonal antibodies are known. In certain embodiments, the antibody and/or an antigen binding part thereof is a purified antibody and/or a binding part thereof.

In certain embodiments, the antibody as described herein has an isotype selected from the group consisting of IgG1, IgG2a, IgG2b, IgG3, IgM and IgA. Determination of the isotype of an antibody may be by a known method.

In certain embodiments, the antibody and/or an antigen binding fragment/part thereof is a mouse antibody and/or an antigen binding fragment/part thereof, a human antibody and/or an antigen binding fragment/part thereof, or a humanized antibody and/or an antigen binding fragment/part thereof.

Humanized antibodies, or antibodies adapted for non-rejection by other mammals, may be produced by a suitable method known in the art, including for example resurfacing or CDR grafting. In resurfacing technology, molecular modelling, statistical analysis and mutagenesis are combined to adjust the non-CDR surfaces of variable regions to resemble the surfaces of known antibodies of the target host. Strategies and methods for the resurfacing of antibodies, and other methods for reducing immunogenicity of antibodies within a different host are known, for example as described in U.S. Pat. No. 5,639,641. Humanized forms of the antibodies may also be made by CDR grafting, by substituting the complementarity determining regions of, for example, a mouse antibody, into a human framework domain.

Methods for humanizing antibodies are known. For example, the antibody may be generated as described in U.S. Pat. No. 6,180,370, herein incorporated by reference; WO 92/22653, herein incorporated by reference; Wright et al. (1992) Critical Rev. in Immunol. 12(3,4): 125-168, herein incorporated by reference; and Gu et al. (1997) Thrombosis and Hematocyst 77(4):755-759), herein incorporated by reference.

Humanized antibodies typically have constant regions and variable regions other than the complementarity determining regions (CDRs) derived substantially or exclusively from a human antibody and CDRs derived substantially or exclusively from the non-human antibody of interest.

Techniques developed for the production of “chimeric antibodies”, for example the splicing of mouse antibody genes to human antibody genes to obtain a molecule with appropriate antigen specificity and biological activity, may be performed by a suitable method. For example, chimeric antibodies may be produced as described in Morrison, S. L. et al. (1984) Proc. Natl. Acad. Sci. 81:6851-6855, herein incorporated by reference; Neuberger, M. S. et al. (1984) Nature 312:604-608, herein incorporated by reference; and Takeda, S. et al. (1985) Nature 314:452-454, herein incorporated by reference.

Immunoassays may be used for screening to identify antibodies and/or antigen binding fragments/parts thereof having the desired specificity. Protocols for competitive binding or immunoradiometric assays using either polyclonal or monoclonal antibodies are known.

Antibody molecules and antigen binding fragments/parts thereof may also be produced recombinantly by methods known in the art, for example by expression in E. coli expression systems. For example, a method for the production of recombinant antibodies is as described in U.S. Pat. No. 4,816,567, herein incorporated by reference. Antigen binding fragments/parts may also be produced by phage display technologies, which are known.

In certain embodiments, the HSC70 binding agent comprises a STRO-1 monoclonal antibody and/or an antigen binding fragment/part thereof. STRO-1 antibodies are commercially available, for example as obtained from R&D Systems (MAB1038). STRO-1 antibodies may also be obtained from the Developmental Studies Hybridoma Bank, University of Iowa, Department of Biology, Iowa City, Iowa 52242-1324.

In certain embodiments, the HSC70 binding agent does not substantially bind to HSP70. In certain embodiments, the HSC70 binding agent comprises a dissociation Kd for binding to HSC70 that is at least 10 fold, at least 100 fold, or at least 1000 fold greater than the Kd of binding to HSP70. Methods for determining affinity of binding are known in the art.

In certain embodiments, the HSC70 binding agent binds to cell surface HSC70 and does not substantially bind to cell surface HSP70.

In certain embodiments, the HSC70 binding agent does not substantially bind to a protein encoded by a HSP70 family member selected from one or more of the following genes: HSPA1A, HSPA1B, HSPA1L, HSPA2, HSPA4, HSPA4L, HSPA5, HSPA6, HSPA7, HSPA9, HSPA12A and HSPA14.

In certain embodiments, the method comprises enriching for cells that express HSC70 as a cell surface marker and do not substantially express HSP70 as a cell surface marker. In certain embodiments, the method comprises enriching for cells that express HSC70 as a cell surface marker and express low or reduced levels of HSP70 as a cell surface marker.

Certain embodiments of the present disclosure provide a method of enriching for immature, uncommitted stromal stem cells by enriching for stromal stem cells having surface expression of HSC70, wherein the enriched cells do not substantially express cell surface HSP70.

Certain embodiments of the present disclosure provide a method of enriching for immature, uncommitted stromal stem cells by enriching for stromal stem cells having surface expression of HSC70 but not substantially having surface expression of HSP70.

Certain embodiments of the present disclosure provide a method of enriching for immature, uncommitted stromal stem cells using a HSC70 binding agent, wherein the enriched cells do not substantially express cell surface HSP70.

Certain embodiments of the present disclosure provide a method of enriching for immature, uncommitted stromal stem cells from a population of cells comprising stromal stem cells, the method comprising enriching stromal stem cells from the population of cells using a HSC70 binding agent, wherein the enriched cells do not substantially express cell surface HSP70.

Certain embodiments of the present disclosure provide a method of enriching for immature, uncommitted stromal stem cells from a population of cells comprising stromal stem cells, the method comprising enriching stromal stem cells from the population of cells using a HSC70 binding agent to enrich for immature, uncommitted stromal stem cells, wherein the enriched cells do not substantially express cell surface HSP70.

Certain embodiments of the present disclosure provide a method of enriching for immature, uncommitted stromal stem cells from a population of cells comprising stromal stem cells, the method comprising enriching stromal stem cells having surface expression of HSC70 and not substantially having surface expression of HSP70.

Methods for enriching cells are known in the art. In certain embodiments, the method of enriching cells comprises flow cytometry, cell sorting, magnetic activated cell sorting (for example as commercially used in Miltenyi Biotec MACS Technology or Dynal magnetic bead selection), antibody panning and red-cell rosetting. Other methods for enrichment are contemplated.

In certain embodiments, the method comprises one or more steps of enrichment, such as multiple sorting steps using flow cytometry.

In certain embodiments, the method comprises use of different HSC70 binding agent(s). For example, cells may be initially sorted for STRO-1 binding and subsequently sorted with a different HSC70 binding agent, such as a STRO-1 population of sorted cells being further sorted using a HSC70 binding agent.

In certain embodiments, the method comprising enriching for cells that have reduced expression, or do not substantially express, a marker. In certain embodiments, the method comprising enriching for cells that have reduced expression, or do not substantially express, a cell surface marker. For example, flow cytometry may be used to enrich for cells that are positive for one marker (eg HSC70; STRO-1) and negative for another marker (HSP70).

In certain embodiments, the method comprises enriching for cells that are HSC70+ and HSP70.

In this regard, cells that are HSP70 include, for example, cells that express substantially no detectable cell surface HSP70, and cells that express HSP70 that is substantially reduced to usual levels of HSP70 expressed on the cell surface.

In certain embodiments, the method comprises enriching for cells that are HSC70^{bright} and HSP70^{dim}.

In certain embodiments, the method comprises enriching for cells that express one or more markers selected from the group consisting of Leptin-Receptor, Low affinity NGF-receptor (CD271), EGF-receptor, PDGF-receptor (CD140a,b), IGF-1-receptor, FGF-1,2,3,4-receptor, BMP-receptor, TGF beta-receptor, Alkaline phosphatase, Thrombomodulin, Vimentin, Integrin beta 5, Nestin, Stem cell factor, Collagen type I, Collagen type VI, HSP90, RANKL, STRO-1 antigen, CXCL12, CD10, CD13, CD29, CD44, CD49a,b,d,e,f, CD51, CD54, CD58, CD61, CD73CD90, CD105, CD144, CD146, CD166, CD184, and CD200. Methods for detecting such markers are known in the art.

In certain embodiments, the method comprises enriching for cells that do not substantially express one or more markers selected from the group consisting of c-fms Glycophorin-A, HLA-DR, Von Willebrand Factor, E-Selectin, CD3, CD4, CD11b, CD14, CD18, CD19, CD20, CD31, CD33, CD34, CD38, CD40, CD44, CD45, CD80, CD86, CD117 (c-kit). Methods for confirming the absence of such are markers are known in the art.

In certain embodiments, the method comprises enriching for cells that are STRO-1^{bright} and HSP70^{dim}.

In certain embodiments, the method comprises enriching for cells that are STRO-1⁺ and HSP70⁻.

In certain embodiments, the enriched stromal stem cells comprise cells that are clonogenic. In certain embodiments, the enriched stromal stem cells comprise clonogenic cells. In certain embodiments, the enriched stromal stem cells comprise a substantial proportion of clonogenic cells. In certain embodiments, the enriched stromal stem cells are all substantially clonogenic.

In certain embodiments, the method comprises providing a population of cells comprising stromal stem cells. Populations of cells comprising stromal stem cells are as described herein.

In certain embodiments, the population of cells comprises cells obtained or derived from a subject. In certain embodiments, the population of cells comprises cells obtained or derived from a human subject. In certain embodiments, the population of cells comprises cells obtained or derived from a non-human subject, such as a mammalian subject, a livestock animal (such as a horse, a cow, a sheep, a goat, a pig), a domestic animal (such as a dog or a cat) and other types of animals such as, non-human primates, rabbits, mice and laboratory animals.

In certain embodiments, the population of cells comprises cells obtained, or derived or arising from a source such as cells arising from pluripotent stem cells, cell arising from induced pluripotent stem cells, cells arising from somatic nuclear transfer, and/or adult stem cells.

In certain embodiments, the cells obtained or derived from a subject or another source are processed to permit enrichment of stromal stem cells.

In certain embodiments, the method comprises exposing the population of cells to a HSC70 binding agent. In certain embodiments, the method comprises exposing the population of cells to a binding agent that binds to HSC70 but does not substantially bind to HSP70. Methods for exposing cells to binding agents are known in the art.

In certain embodiments, the method comprises enriching stromal cells from the population of cells using a HSC70 binding agent bound to cells.

Certain embodiments of the present disclosure provide a method of enriching for immature, uncommitted stromal stem cells, the method comprising:
providing a population of cells comprising stromal stem cells;
exposing the population of cells to a HSC70 binding agent; and
enriching stromal cells from the population of cells using the HSC70 binding agent bound to cells.

In certain embodiments, the enrichment of stromal stem cells comprises detecting cells that express cell surface HSC70. In certain embodiments, the method comprises enriching stromal cells from the population of cells by detecting cells that express HSC70. In certain embodiments, the method comprises using a HSC70 binding agent to detect cells that express cell surface HSC70.

In certain embodiments, the enrichment of stromal stem cells comprises detecting cells that express HSC70 as a cell surface marker. In certain embodiments, the method comprises enriching stromal cells from the population of cells by detecting cells that express HSC70 as a cell surface marker. In certain embodiments, the method comprises using a HSC70 binding agent to detect cells that express HSC70 as a cell surface marker. In certain embodiments, the method comprising enriching stromal cells from the population of cells by detecting cells bound to a HSC70 binding agent.

Certain embodiments of the present disclosure provide a method of enriching for immature, uncommitted stromal stem cells from a population of cells comprising stromal stem cells, the method comprising enriching stromal cells from the population of cells by detecting cells that express HSC70 as a cell surface marker.

Certain embodiments of the present disclosure provide a method of enriching for immature, uncommitted stromal stem cells from a population of cells comprising stromal stem cells, the method comprising enriching stromal cells from the population of cells expressing HSC70 as a cell surface marker and thereby enriching for immature, uncommitted stromal stem cells.

Certain embodiments of the present disclosure provide a method of enriching for immature, uncommitted stromal stem cells from a population of cells comprising stromal stem cells, the method comprising enriching stromal cells from the population of cells by detecting cells bound to a HSC70 binding agent.

Certain embodiments of the present disclosure provide a method of enriching for immature, uncommitted mesenchymal stem cells (MSCs).

Certain embodiments of the present disclosure provide a method of enriching for immature, uncommitted MSCs, the method comprising enriching stromal stem cells using a HSC70 binding agent.

Methods for enriching stromal stem cells are as described herein. HSC70 binding agents and methods for use of the binding agents are as described herein.

Certain embodiments of the present disclosure provide stromal stem cells enriched by a method as described herein.

In certain embodiments, the enriched stromal stem cells comprise clonogenic cells.

In certain embodiments, the enriched stromal stem cells are HSC70⁺ and HSP70⁻. In certain embodiments, the enriched stromal cells are HSC70^{bright} and HSP70^{dim}. In certain embodiments, the enriched stromal stem cells are STRO-1⁺ and HSP70⁻. In certain embodiments, the enriched stromal cells are STRO-1^{bright} and HSP70^{dim}.

In certain embodiments, the enriched stromal stem cells comprise immature, uncommitted MSCs.

In certain embodiments, the enriched stromal cells are HSC70- and HSP70′. In certain embodiments, the enriched stromal cells are HSC70^{bright} and HSP70^{dim}.

Certain embodiments of the present disclosure provide a population of cells comprising stromal stem cells enriched by a method as described herein.

Certain embodiments of the present disclosure provide a method of enriching for immature, uncommitted mesenchymal stem cells from a population of cells comprising stromal stem cells, the method comprising enriching immature and/or precursor mesenchymal stem cells from the population of cells using a HSC70 binding agent.

Certain embodiments of the present disclosure provide immature, uncommitted mesenchymal stem cells enriched using a method as described herein.

Certain embodiments of the present disclosure provide a method of isolating immature, uncommitted stromal stem cells.

Certain embodiments of the present disclosure provide a method of isolating immature, uncommitted stromal stem cells from a population of cells comprising stromal stem cells, the method comprising isolating stromal stem cells expressing cell surface HSC70 from the population of cells.

Certain embodiments of the present disclosure provide a method of isolating immature, uncommitted stromal stem cells from a population of cells comprising stromal stem cells, the method comprising isolating stromal stem cells from the population of cells using a HSC70 binding agent.

The term “isolating” or the related terms “isolate” or “isolated” refer to a process whereby a species, such as a cell, a nucleic acid or a polypeptide, has been separated (partially or completely) from its natural or original environment.

For example, an isolated cell may be in a substantially purified state, or be a cell in a population of other cells.

Methods for isolating cells are known in the art. In certain embodiments, the method of isolating cells comprises one or more of flow cytometry, cell sorting magnetic activated cell sorting (for example as commercially used in Miltenyi Biotec MACS Technology or Dynal magnetic bead selection), antibody panning and red-cell rosetting. Other methods for isolating cells are contemplated.

In certain embodiments the stromal stem cells comprise mesenchymal stem cells. Other types of cells are contemplated.

In certain embodiments, the population of cells comprises stromal stem cells obtained, derived and/or arising from, placenta, umbilical cord, umbilical cord blood, tooth bud tissue, dentine/pulp tissue, periodontal ligament, gingival, skin, hair, follicle, amniotic fluid, adipose tissue, smooth muscle, skeletal muscle, Rendon, ligament, bong, cartilage, bone marrow and/or peripheral blood. Other types of sources are contemplated. Methods for obtaining stem cells from such sources are known in the art.

In certain embodiments, the population of cells comprises stromal stem cells arising from pluripotent stem cells, induced pluripotent stem cells, cells arising from somatic nuclear transfer, and/or adult stem cells. Methods for obtaining stem cells from such sources are known in the art.

In certain embodiments, the isolation of the stromal stem cells results in a population of cells whereby the immature, uncommitted stromal stem cells comprises at least 1%, at least 2%, at least 5%, at least 10%, at least 20%, at least 30%, at least 40%, at least 50%, at least 60%, at least 70%, at least 80%, at least 90%, at least 95% or at least 99% of the total cells in the population. In certain embodiments, the isolation of the stromal stem cells results in a population of cells whereby the immature, uncommitted stromal stem cells comprises about 50% of the total cells in the population. Other levels are contemplated.

In certain embodiments, the isolating comprises isolating stromal stem cells from the population of cell so that the isolated immature, uncommitted stromal stem cells comprise substantially the only cells present.

Isolating cells that express HSC70 as a cell surface marker is as described herein. For example, HSC70 binding agents, and their use, are as described herein.

In certain embodiments, the isolating comprises isolation of immature, uncommitted stromal stem cells from a human subject. In certain embodiments, the isolating comprises isolation of immature, uncommitted stromal stem cells from a non-human subject.

In certain embodiment, a HSC70 binding agent is used to isolate immature, uncommitted stromal stem cells.

In certain embodiments, a HSC70 binding agent is used to isolate immature, uncommitted stromal stem cells from a human subject. In certain embodiments, a HSC70 binding agent is used to isolate immature, uncommitted stromal stem cells from a non-human subject.

In certain embodiments, the HSC70 binding agent comprises one or more of an antibody and/or an antigen binding part thereof, a small molecule, a nucleic acid, an aptamer, a polypeptide, a protein, ligand or a ligand mimetic. Other types of agents are contemplated.

In certain embodiments, the method comprises isolating cells that express HSC70 as a cell surface marker. In certain embodiments, the method comprises isolating cells with surface expressed HSC70.

In certain embodiments, the method comprises binding of a HSC70 binding agent to cell surface HSC70. In certain embodiments, the HSC70 binding agent binds to cell surface HSC70. Methods for detecting the binding of agents to cell surface markers are known in the art.

In certain embodiments, the HSC70 binding agent comprises a ligand to the HSC70 protein. Examples of ligands to HSC70 are described herein.

In certain embodiments, the HSC70 binding agent comprises an anti-HSC70 antibody, and/or an antigen binding part thereof. In certain embodiments, the HSC70 binding agent comprises a soluble portion of a ligand to a HSC70 ligand.

In certain embodiments, the method comprises using a HSC70 binding agent bound to cells to enrich cells.

In certain embodiments, the HSC70 binding agent comprises all or part of a EWI-2 protein (CD316) and/or a variant thereof. In certain embodiments, the HSC70 ligand comprises all or part of a EWI-2 protein (CD316) and/or a variant thereof. In certain embodiments, the HSC70 ligand comprises all or part of an extracellular domain/region of a EWI-2 protein (CD316) and/or a variant thereof. In certain embodiments, the HSC70 ligand comprises all or part of an extracellular domain/region of a EWI-2 protein (CD316) and/or a variant thereof and a detectable tag, such as a sEWI-2-hIg protein with four extracellular domains, including amino acids 1 to 574 fused to the J-CH₂—CH₃ domains of the hIgG heavy chain, as described in Kettner et al. (2007) Mol Cell Biol. 27(21):7718-26.

In certain embodiments, the HSC70 binding agent comprises an antibody and/or an antigen binding part thereof, such as STRO-1. Antibodies, and antigen binding parts thereof, are as described herein.

In certain embodiments, the HSC70 binding agent comprises an antibody, and/or antigen binding part thereof, to a HSC70 protein. In certain embodiments, the HSC70 binding agent comprises an antibody to human HSC70.

In certain embodiments, the HSC70 binding agent comprises a STRO-1 monoclonal antibody and/or an antigen binding part/fragment thereof. STRO-1 antibodies are commercially available.

In certain embodiments, the HSC70 binding agent does not substantially bind to HSP70. In certain embodiments, the HSC70 binding agent binds to cell surface HSC70 and does not substantially bind to cell surface HSP70.

In certain embodiments, the HSC70 binding agent does not substantially bind to a protein encoded by a HSP70 family member selected from one or more of the following genes: HSPA1A, HSPA1B, HSPA1L, HSPA2, HSPA4, HSPA4L, HSPA5, HSPA6, HSPA7, HSPA9, HSPA12A and HSPA14.

In certain embodiments, the method comprises isolating cells that express HSC70 as a cell surface marker and do not substantially express HSP70 as a cell surface marker. In certain embodiments, the method comprises isolating cells that express HSC70 as a cell surface marker and express low or reduced levels of HSP70 as a cell surface marker.

In certain embodiments, the method comprises isolating cells that are HSC70 and HSP70′.

In certain embodiments, the method comprises isolating cells that are HSC70^{bright} and HSP70^{dim}.

In certain embodiments, the method comprises isolating cells that express one or more markers selected from the group consisting of Leptin-Receptor, Low affinity NGF-receptor (CD271), EGF-receptor, PDGF-receptor (CD140a,b), IGF-1-receptor, FGF-1,2,3,4-receptor, BMP-receptor, TGF beta-receptor, Alkaline phosphatase, Thrombomodulin, Vimentin, Integrin beta 5, Nestin, Stem cell factor, Collagen type I, Collagen type VI, HSP90, RANKL, STRO-1 antigen, CXCL12, CD10, CD13, CD29, CD44, CD49a,b,d,e,f, CD51, CD54, CD58, CD61, CD73CD90, CD105, CD144, CD146, CD166, CD184, and CD200. Methods for detecting such markers are known in the art.

In certain embodiments, the method comprises isolating cells that do not substantially express one or more markers selected from the group consisting of c-fms Glycophorin-A, HLA-DR, Von Willebrand Factor, E-Selectin, CD3, CD4, CD11b, CD14, CD18, CD19, CD20, CD31, CD33, CD34, CD38, CD40, CD44, CD45, CD80, CD86, CD117 (c-kit). Methods for confirming the absence of such are markers are known in the art.

In certain embodiments, the isolated stromal stem cells are clonogenic. In certain embodiments, the isolated stromal stem cells comprise one or more cells that are clonogenic. In certain embodiments, the isolated stromal stem cells comprise clonogenic cells. In certain embodiments, the isolated stromal stem cells comprise a substantial proportion of clonogenic cells. In certain embodiments, the isolated stromal stem cells are all substantially clonogenic.

In certain embodiments, the method comprises providing a population of cells comprising stromal stem cells. Populations of cells comprising stromal stem cells are as described herein.

In certain embodiments, the population of comprises cells obtained or derived from a subject. In certain embodiments, the population of cells comprises cells obtained or derived from a human subject. In certain embodiments, the population of cells comprises cells obtained or derived from a non-human subject, a mammalian subject, a livestock animal (such as a horse, a cow, a sheep, a goat, a pig), a domestic animal (such as a dog or a cat) and other types of animals such as, non-human primates, rabbits, mice and laboratory animals. Methods for obtaining cells from such sources are known in the art. Other types of subjects are contemplated.

In certain embodiments, the population of comprises cells obtained or derived from a source such as cells arising from pluripotent stem cells, cell arising from induced pluripotent stem cells, cells arising from somatic nuclear transfer, and/or adult stem cells. Methods for obtaining cells from such sources are known in the art.

In certain embodiments, the cells obtained or derived from a subject or another source are processed to permit isolation of stromal stem cells.

In certain embodiments, the method comprises exposing the population of cells to a HSC70 binding agent. Methods for exposing cells to binding agents are known in the art.

In certain embodiments, the method comprises isolating immature, uncommitted stromal cells from the population of cells using a HSC70 binding agent bound to cells.

Certain embodiments of the present disclosure provide a method of isolating immature, uncommitted stromal stem cells from a population of cells comprising stroma stem cells, the method comprising isolating stromal cells from the population of cells using a HSC70 binding agent bound to cells.

Certain embodiments of the present disclosure provide a method of isolating immature, uncommitted stromal stem cells, the method comprising:
providing a population of cells comprising stromal stem cells;
exposing the population of cells to a HSC70 binding agent; and
isolating stromal cells from the population of cells using the HSC70 binding agent bound to cells.

Certain embodiments of the present disclosure provide a method of isolating immature, uncommitted stromal stem cells from a population of cells comprising stromal stem cells, the method comprising isolating stromal cells from the population of cells by detecting cells that express HSC70 as a cell surface marker.

Certain embodiments of the present disclosure provide a method of isolating immature, uncommitted stromal stem cells from a population of cells comprising stromal stem cells, the method comprising isolating stromal cells from the population of cells by detecting cells bound to a HSC70 binding agent.

Certain embodiments of the present disclosure provide a method of isolating immature, uncommitted stromal stem cells, the method comprising isolating stromal stem cells using a HSC70 binding agent.

Certain embodiments of the present disclosure provide one or more immature, uncommitted stromal stem cells isolated by a method as described herein.

In certain embodiments, the one or more cells do not substantially express HSP70 as a cell surface marker. In certain embodiments, the one or more cells are HSP70⁻.

In certain embodiments, the one or more stromal stem cells are HSP70⁻ or HSP70^{dim}.

Certain embodiments of the present disclosure provide a method of isolating immature, uncommitted stromal stem cells from a population of cells comprising stroma stem cells, the method comprising isolating stromal cells from the population of cells using a HSC70 binding agent bound to cells, wherein the isolated cells do not substantially express cell surface HSP70.

Certain embodiments of the present disclosure provide one or more isolated immature, uncommitted stromal stem cells.

In certain embodiments, the one or more stromal stem cells comprise one or more immature, uncommitted MSCs.

In certain embodiments, the one or more isolated stromal cells comprise one or more clonogenic cells.

Certain embodiments of the present disclosure provide a method of isolating immature, uncommitted mesenchymal stem cells from a population of cells comprising stromal stem cells, the method comprising isolating immature and/or precursor mesenchymal stem cells from the population of cells using a HSC70 binding agent.

Certain embodiments of the present disclosure provide one or more isolated stromal stem cells, the stromal stem cells expressing HSC70 as a cell surface marker and not substantially expressing HSP70 as a cell surface marker.

Methods for identifying stromal stem cells are as described herein, including identifying the presence and/or absence of markers that are indicative of the cells.

Methods for detecting HSC70 and HSP70 as cell surface markers are as described herein.

In certain embodiments, the one or more stromal stem cells are part of a mixture of one or more other cells, such as a population of cells, as described herein. In certain embodiments, the one or more stromal stem cells comprise a plurality of cells.

Methods for producing stromal stem cells that express HSC70 as a cell surface marker and not substantially expressing HSP70 as a cell surface marker are as described herein.

Certain embodiments of the present disclosure provide one or more isolated stromal stem cells, the stromal stem cells comprising HSC70⁺ and HSP70⁻ cell surface markers.

Methods for identifying stromal stem cells are as described herein, including identifying the presence and/or absence of markers that are indicative of the cells.

Methods for detecting HSC70 and HSP70 as cell surface markers are as described herein. Methods for determining whether cells are HSC70⁺ and HSP70⁻ are as described herein.

In certain embodiments, the one or more stromal stem cells are part of a mixture of one or more other cells, such as a population of cells, as described herein. In certain embodiments, the one or more stromal stem cells comprise a plurality of cells.

Methods for producing stromal stem cells that are HSC70⁺ and HSP70⁻ are as described herein.

Certain embodiments of the present disclosure provide one or more isolated stromal stem cells, the stromal stem cells comprising HSC70^{bright} and HSP70^{dim} cell surface markers.

Methods for identifying stromal stem cells are as described herein, including identifying the presence and/or absence of markers that are indicative of the cells.

Methods for detecting HSC70 and HSP70 as cell surface markers are as described herein. Methods for determining whether cells are HSC70^{bright} and HSP70^{dim} are as described herein.

In certain embodiments, the one or more stromal stem cells are part of a mixture of one or more other cells, such as a population of cells, as described herein. In certain embodiments, the one or more stromal stem cells comprise a plurality of cells.

Methods for producing stromal stem cells that are HSC70^{bright} and HSP70^{dim} are as described herein.

Certain embodiments of the present disclosure provide one or more isolated stromal stem cells, the stromal stem cells expressing a STRO-1 antigen as a cell surface marker and not substantially expressing HSP70 as a cell surface marker.

In this regard, the term “STRO-1⁺” or variants such as “STRO-1^{bright}”, “STRO-1^{dim}” and “STRO-1⁻” refers to the degree to which the STRO-1 antibody binds to the cell surface of stromal stem cells. The degree of STRO-1 antibody binding is directly related to the density of cell surface antigen recognised by the STRO-1 antibody.

In this regard, it has been found that a cell population isolated using STRO-1 includes cells that also express HSP70 on the cell surface, and that the CFU-F activity is limited to the STRO-1⁺/HSP70⁻ population. Thus, such cells have improved clonogenic activity over STRO-1⁺ cells.

Methods for identifying stromal stem cells are as described herein, including identifying the presence and/or absence of markers that are indicative of the cells.

STRO-1 antibodies, and their use, are as described herein.

Methods for using a STRO-1 antigen and HSP70 as cell surface markers are as described herein.

In certain embodiments, the one or more stromal stem cells are part of a mixture of one or more other cells, such as a population of cells, as described herein. In certain embodiments, the one or more stromal stem cells comprise a plurality of cells.

Methods for producing stromal stem cells that express a STRO-1 antigen as a cell surface marker and do not substantially express HSP70 as a cell surface marker are as described herein.

Certain embodiments of the present disclosure provide one or more isolated stromal stem cells, the stromal stem cells comprising STRO-1⁺ and HSP70⁻ cell surface markers.

Methods for identifying stromal stem cells are as described herein, including identifying the presence and/or absence of markers that are indicative of the cells.

Methods for detecting a STRO-1 antigen and HSP70 as a cell surface marker are as described herein. Methods for determining whether cells are STRO-1⁺ and HSP70⁻ are as described herein.

In certain embodiments, the one or more stromal stem cells are part of a mixture of one or more other cells, such as a population of cells, as described herein. In certain embodiments, the one or more stromal stem cells comprise a plurality of cells.

Methods for producing stromal stem cells that are STRO-1⁺ and HSP70⁻ are as described herein.

Certain embodiments of the present disclosure provide one or more isolated stromal stem cells, the stromal stem cells comprising STRO-1^{bright} and HSP70^{dim} cell surface markers.

Methods for identifying stromal stem cells are as described herein, including identifying the presence and/or absence of markers that are indicative of the cells.

Methods for detecting a STRO-1 antigen and HSP70 as a cell surface marker are as described herein. Methods for determining whether cells are STRO-1^{bright} and HSP70^{dim} are as described herein.

In certain embodiments, the one or more stromal stem cells are part of a mixture of one or more other cells, such as a population of cells, as described herein. In certain embodiments, the one or more stromal stem cells comprise a plurality of cells.

Methods for producing stromal stem cells that are STRO-1^{bright} and HSP70^{dim} are as described herein.

Certain embodiments of the present disclosure provide a population of cells comprising one or more isolated stromal stem cells as described herein.

Certain embodiments of the present disclosure provide a method of enriching for immature, uncommitted stromal stem cells from a population of cells comprising stromal stem cells, the method comprising enriching for stromal stem cells expressing a STRO-1 antigen as a cell surface marker and not substantially expressing HSP70 as a cell surface marker from the population of cells.

Certain embodiments of the present disclosure provide a method of enriching for stromal stem cells from a population of cells comprising stromal stem cells, the method comprising enriching for STRO-1⁺ HSP70⁻ stromal stem cells from the population of cells.

Methods for enriching stromal stem cells are as described herein. STRO-1 antibodies, and their use, are as described herein.

Methods for detecting and enriching cells that are HSP70⁻ are as described herein.

Certain embodiments of the present disclosure provide a method of enriching for stromal stem cells from a population of cells comprising stromal stem cells, the method comprising enriching for STRO-1^{bright} HSP70^{dim} stromal stem cells from the population of cells.

Certain embodiments of the present disclosure provide a method of isolating immature, uncommitted stromal stem cells from a population of cells comprising stromal stem cells, the method comprising isolating stromal stem cells expressing a STRO-1 antigen as a cell surface marker and not substantially expressing HSP70 as a cell surface marker from the population of cells.

Certain embodiments of the present disclosure provide a method of isolating immature, uncommitted stromal stem cells from a population of cells comprising stromal stem cells, the method comprising isolating STRO-1⁺ HSP70⁻ stromal cells from the population of cells.

Methods for isolating stromal stem cells are as described herein. STRO-1 antibodies, and their use, are as described herein.

Methods for detecting and isolating cells that are HSP70⁻ are as described herein.

Certain embodiments of the present disclosure provide a method of isolating immature, uncommitted stromal stem cells from a population of cells comprising stromal stem cells, the method comprising isolating STRO-1^{bright} HSP70^{dim} stromal stem cells from the population of cells.

Certain embodiments of the present disclosure provide a method of identifying an immature, uncommitted stromal stem cell.

Certain embodiments of the present disclosure provide use of HSC70 as a marker of an immature, uncommitted stromal stem cell.

Certain embodiments of the present disclosure provide a method of identifying an immature, uncommitted stromal stem cell, the method comprising identifying a stromal stem cell that expresses a HSC70 cell surface marker.

Methods for identifying stromal stem cells with specific cell surface markers are as described herein.

In certain embodiments, the method further comprises identifying a cell that does not substantially express HSP70 cell surface marker.

HSP70 cell surface markers, and their identification, are as described herein.

In certain embodiments, the method comprises identifying an immature, uncommitted MSC.

Certain embodiments of the present disclosure provide a method of identifying an immature, uncommitted MSC, the method comprising identifying a mesenchymal stem cell that expresses a HSC70 cell surface marker.

Certain embodiments of the present disclosure provide a method of identifying an agent for enriching, isolating and/or identifying a stromal stem cell.

Certain embodiments of the present disclosure provide a method of identifying an agent for enriching, isolating and/or identifying an immature, uncommitted stromal stem cell, the method comprising:
determining whether a candidate agent binds to HSC70 on a stromal stem cell; and
identifying the candidate agent as an agent for enriching, isolating and/or
identifying an immature, uncommitted, stromal stem cell.

Methods for determining the binding of agents to HSC70 are known in the art, examples of which are also as described herein.

In certain embodiments, the candidate agent is a ligand of HSC70. In certain embodiments, the candidate agent is an antibody and/or an antigen binding part thereof.

Methods for determining the ability of a candidate agent to enrich, isolate or identify immature, uncommitted, stromal cells are as described herein.

In certain embodiments, the method further comprises determining whether the candidate agent does not bind substantially to HSP70 on the stromal stem cell.

Methods for determining the binding of agents to HSP70 are known in the art, examples of which are also described herein.

In certain embodiments, the method comprises identifying an agent for enriching, isolating and/or identifying immature, uncommitted MSCs.

Certain embodiments of the present disclosure provide a method of identifying an agent for enriching, isolating and/or identifying an immature, uncommitted mesenchymal stem cell, the method comprising:
determining whether a candidate agent binds to HSC70 on a stromal stem cell; and
identifying the candidate agent as an agent for enriching, isolating and/or
identifying an immature, uncommitted mesenchymal stem cell.

Certain embodiments of the present disclosure provide isolated and/or non-naturally occurring polypeptides, and agents that comprise such polypeptides.

Certain embodiments of the present disclosure provide an isolated and/or non-naturally occurring polypeptide comprising one or more of the following amino acid sequences: (i) KDISENKRAVRRLR (SEQ ID NO. 2), ISENKRAVRRLARTA (SEQ ID NO. 3), ISENKRAVRRLAR (SEQ ID NO. 4) and/or a variant of any of the aforementioned amino acid sequences. In certain embodiments, the isolated and/or non-naturally occurring polypeptide consists of an amino acid sequence one of SEQ ID NOs 2 to 4 and/or a variant of any of the aforementioned sequences, such as a variant consisting of a deletion of one or more amino acids from either or both of the NH₂ or COOH termini.

Certain embodiments of the present disclosure provide an isolated and/or a non-naturally occurring polypeptide comprising the amino acid sequence according to SEQ ID NO.4 and/or a variant thereof.

In certain embodiments, the polypeptide consists of the amino acid sequence according to SEQ ID NO. 2, SEQ ID NO. 3 or SEQ ID NO.4, or a variant of the aforementioned amino acid sequences.

In certain embodiments, the polypeptide comprises a polypeptide comprising amino acids 204-393 of hHSC70 and/or a variant thereof. The equivalent region in HSC70 in other species can be readily determined by a person skilled in the art.

In certain embodiments, the polypeptide comprises a STRO-1 binding epitope present in a region spanning amino acids 204-393 of hHSC70. Equivalent epitopes in other species can be readily determined by a person skilled in the art.

Such polypeptides may be useful, for example, for blocking STRO-1 antibody binding to a target and/or for raising antibodies.

Methods for producing oligopeptides and polypeptides are known in the art, such as chemical synthesis or by recombinant DNA technology, for example as described generally in Green M R and Sambrook J, Molecular Cloning: A Laboratory Manual (4th edition), Cold Spring Harbor Laboratory Press, 2012, herein incorporated by reference, and Ausubel et al., Current Protocols in Molecular Biology (2011), John Wiley & Sons, Inc.

Certain embodiments of the present disclosure provide a method of inhibiting or blocking STRO-1 binding. Methods for assessing binding are known in the art.

Certain embodiments of the present disclosure provide a method of inhibiting or blocking STRO-1 antibody binding to a cell, the method comprising using a polypeptide and/or agent as described herein to block the binding of STRO-1 to the cell.

Methods for using agents to block binding and/or as antibody antagonists are known in the art. For example, a polypeptide comprising the first 393 amino acids of HSC70 can be used to block STRO-1 binding.

Certain embodiments of the present disclosure provide a STRO-1 antagonist, the antagonist comprising one or more of the following amino acid sequences: (i) KDISENKRAVRRLR (SEQ ID NO. 2), ISENKRAVRRLARTA (SEQ ID NO. 3), ISENKRAVRRLAR (SEQ ID NO. 4) and/or a functional variant of any of the aforementioned amino acid sequences. Variants are as described herein. In certain embodiments, the antagonist consists of an amino acid sequence one of SEQ ID NOs 2 to 4 and/or a variant of any of the aforementioned sequences, such as a variant consisting of a deletion of one or more amino acids from either or both of the NH₂ or COOH termini.

Certain embodiments of the present disclosure provide a method of inhibiting STRO-1 antibody binding to a cell, the method comprising using an agent comprising an amino acid sequence according to SEQ ID NO.4 and/or a variant thereof.

Certain embodiments of the present disclosure provide a STRO-1 antagonist comprising an amino acid sequence according to SEQ ID NO. 4 and/or a functional variant thereof.

In certain embodiments, the variant has at least 70%, at least 80%, at least 85%, at least 90%/o, at least 95%, at least 96%, at least 97%, at least 98% or at least 99%/o identity with one or more of SEQ ID NOs. 2 to 4. In certain embodiments, the variant has at least 70%, at least 80%, at least 85%, at least 90%, at least 95%, at least 96%, at least 97%, at least 98% or at least 99^{%}, homology with one or more of SEQ ID NOs. 2 to 4.

Certain embodiments of the present disclosure provide a method of inhibiting STRO-1 binding, the method comprising use of a polypeptide as described herein and/or an antagonist as described herein.

In certain embodiments, the method comprises use of a polypeptide comprising the amino acid sequence according to SEQ ID NO.4 and/or a variant thereof.

In certain embodiments, the method comprises use of a STRO-1 antagonist comprising an amino acid sequence according to SEQ ID NO. 4 and/or a functional variant thereof.

Certain embodiments of the present disclosure provide a method of inhibiting STRO-1 binding to a cell, the method comprising using an agent comprising an amino acid sequence according to SEQ ID NO.4 and/or a functional variant thereof to inhibit the binding of STRO-1 to the cell.

Certain embodiments of the present disclosure provide a method of identifying a HSC70 binding agent. Such agents are useful, for example, for use in enriching and/or isolating cells.

HSC70 binding agents are as described herein. Methods for determining binding of agents are known in the art.

In certain embodiments, the HSC70 binding agent does not bind substantially to HSP70.

In certain embodiments, the HSC70 binding agent comprises a dissociation Kd for binding to HSC70 that is at least 10 fold, at least 100 fold, or at least 1000 fold greater than the Kd of binding to HSP70. Methods for determining affinity of binding are known in the art.

Certain embodiments of the present disclosure provide a method of identifying a HSC70 binding agent, the method comprising identifying an agent that binds to HSC70 and does not substantially bind to HSP70.

Certain embodiments of the present disclosure provide a method of producing an antibody, the method comprising raising an antibody against a polypeptide as described herein.

Methods for producing antibodies are known in the art and are as described herein.

Certain embodiments of the present disclosure provide use of a polypeptide as described herein for raising an antibody. Certain embodiments of the present disclosure provide use of a polypeptide as described herein for immunizing an animal.

Certain embodiments of the present disclosure provide an agent comprising a polypeptide as described herein.

The term “polypeptide” as used herein refers to oligo- and poly-peptides and refers to substances comprising for example two or more, 3 or more, 4 or more, 5 or more, 6 or more, 7 or more, 8 or more, 9 or more, 10 or more, 20 or more, 30 or more, 40 or more or 50 or more amino acids joined covalently by peptide bonds. The term “protein” typically refers to large polypeptide, but in general the terms “polypeptides” and “proteins” are synonyms and are used interchangeably herein.

In certain embodiments, the polypeptides described herein are isolated. For example, an isolated polypeptide may be in a partially purified state, or a substantially purified state, being substantially free of other substances with which it is associated in nature or in vivo. In certain embodiments, the polypeptides as described herein are non-naturally occurring.

Polypeptides described herein may be isolated from biological samples (such as tissue or cell homogenates), may be expressed recombinantly in a multiplicity of pro- or eukaryotic expression systems, or synthesized by known chemical means.

The term “variant” of a polypeptide or of an amino acid sequence refers to one or more of amino acid insertion variants, amino acid deletion variants, amino acid substitution variants, and amino acid modification variants (natural and/or synthetic).

For example, amino acid insertion variants may comprise amino- and/or carboxy-terminal fusions and also insertions of single or two or more amino acids in a particular amino acid sequence. In the case of amino acid sequence variants having an insertion, one or more amino acid residues may be inserted into a particular site in an amino acid sequence, although random insertion with appropriate screening of the resulting product is also possible.

Amino acid deletion variants are characterized by the removal of one or more amino acids from the sequence.

Amino acid fusion variants are characterized by the addition of one or more amino acids from to sequence, which typically are NH₂-terminal fusions, COOH-terminal fusions and/or internal fusions.

Amino acid substitution variants are characterized by at least one residue in the sequence being removed and one or more other residues being inserted in its place.

Amino acid changes in variants may be non-conservative and/or conservative amino acid changes, i.e., substitutions of similarly charged or uncharged amino acids. A conservative amino acid change typically involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cysteine, serine, threonine, tyrosine) amino acids. Phenylalanine, tryptophan, and tyrosine are sometimes classified jointly as aromatic amino acids.

In certain embodiments, the degree of similarity, for example identity or homology, between a given amino acid sequence and an amino acid sequence which is a variant of said given amino acid sequence will be at least 70%, at least 80° 6, at least 85%, at least 90%, at least 95%, at least 96%, at least 97%, at least 98% or at least 99%.

In certain embodiments, the degree of identity between a given amino acid sequence and an amino acid sequence which is a variant of said given amino acid sequence will be at least 70%, at least 80° 6, at least 85%, at least 90%, at least 95%, at least 96%, at least 97%, at least 98% or at least 99%.

In certain embodiments, the degree of homology between a given amino acid sequence and an amino acid sequence which is a variant of said given amino acid sequence will be at least 70° 6, at least 80%, at least 85%, at least 90°/%, at least 95%, at least 96%, at least 97%, at least 98% or at least 99%.

The degree of similarity or identity may be for a region of at least about 10, at least 20, at least 40, at least 60, at least 80, at least 100, at least 120, at least 140, at least 160, or at least 200 amino acids.

The polypeptides and amino acid variants described herein may be readily prepared with the aid of known peptide synthesis techniques such as, for example, by solid phase synthesis and similar methods or by recombinant DNA manipulation. The manipulation of DNA sequences for preparing proteins and peptides having substitutions, insertions or deletions, is described for example in Green M R and Sambrook J, Molecular Cloning: A Laboratory Manual (4th edition), Cold Spring Harbor Laboratory Press, 2012, herein incorporated by reference. Expression, purification and analysis of expressed proteins may be performed by methods known in the art, for example as described in “The Recombinant Protein Handbook—Protein Amplification and Simple Purification, 18-1142-75, Amersham Pharma Biotech, edition AA.

The polypeptides as described herein also include modified forms of polypeptides. Such modifications include any chemical modification and comprise single or multiple substitutions, deletions and/or additions of any molecules associated with the protein or peptide, such as carbohydrates, lipids and/or proteins or peptides.

In certain embodiments, a polypeptide as described herein is a non-naturally occurring polypeptide. In certain embodiments, a non-naturally occurring peptide is a synthetic peptide or an isolated peptide. In certain embodiments, a non-naturally occurring peptide is produced by recombinant DNA technology. In certain embodiments, a polypeptide as described herein is produced by chemical synthesis.

Methods for isolating and/or producing polypeptides and proteins are known, and are as described generally in Green M R and Sambrook J, Molecular Cloning: A Laboratory Manual (4th edition), Cold Spring Harbor Laboratory Press, 2012, herein incorporated by reference, and Ausubel et al., Current Protocols in Molecular Biology (2011), John Wiley & Sons, Inc., herein incorporated by reference.

Certain embodiments of the present disclosure provide an isolated agent comprising a HSC70 ligand, wherein the agent does not substantially bind to HSP70. Methods for determining the ability of a ligand to bind to HSC70 and HSP70 are known in the art.

Certain embodiments of the present disclosure provide a non-naturally agent comprising a HSC70 ligand, wherein the agent does not substantially bind to HSP70.

Certain embodiments of the present disclosure provide an isolated, non-naturally agent comprising a HSC70 ligand, wherein the agent does not substantially bind to HSP70.

Examples of agents include one or more of an antibody and/or an antigen binding part thereof, a small molecule, a nucleic acid, an aptamer, a polypeptide, a protein, ligand or a ligand mimetic.

Methods for determining the ability of an agent to bind to HSC70 and HSP70 are known in the art.

In certain embodiments, the agent comprises a detectable tag. Detectable tags are as described herein.

In certain embodiments, the agent comprises a polypeptide. Methods for producing polypeptides, including a variant of a polypeptide, are known in the art. For example, the agent may comprise a polypeptide produced by recombinant means.

In certain embodiments, the agent comprises all or part of an extracellular region of a EWI-2 protein, and/or a variant thereof. The accession number for human EWI-2 is NCBI Reference Sequence: NC_000001.10. The gene in other species may be identified by a person skilled in the art. The amino acid sequence of the extracellular domain of human EWI-2 protein (SEQ ID NO. 5) is as follows:

(SEQ ID NO: 5)
MGALRPTLLPPSLPLLLLLMLGMGCWAREVLVPEGPLYRVAGTAVSISCN
VTGYEGPAQQNFEWFLYRPEAPDTALGIVSTKDTQFSYAVFKSRVVAGEV
QVQRLQGDAVVLKIARLQAQDAGIYECHTPSTDTRYLGSYSGKVELRVLP
DVLQVSAAPPGPRGRQAPTSPPRMTVHEGQELALGCLARTSTQKHTHLAV
SFGRSVPEAPVGRSTLQEVVGIRSDLAVEAGAPYAERLAAGELRLGKEGT
DRYRMVVGGAQAGDAGTYHCTAAEWIQDPDGSWAQIAEKRAVLAHVDVQT
LSSQLAVTVGPGERRIGPGEPLELLCNVSGALPPAGRHAAYSVGWEMAPA
GAPGPGRLVAQLDTEGVGSLGPGYEGRHIAMEKVASRTYRLRLEAARPGD
AGTYRCLAKAYVRGSGTRLREAASARSRPLPVHVREEGVVLEAVAWLAGG
TVYRGETASLLCNISVRGGPPGLRLAASWWVERPEDGELSSVPAQLVGGV
GQDGVAELGVRPGGGPVSVELVGPRSHRLRLHSLGPEDEGVYHCAPSAWV
QHADSWYQAGSARSGPVTVYPYM.

In certain embodiments, the agent comprises a part of the extracellular domain of a EWI-2 protein that binds to HSC70, and/or variant thereof. In certain embodiments, the agent comprises a part of the extracellular domain of human EWI-2 that binds to HSC70, and/or a variant thereof. Methods for identifying a EWI-2 protein from other species are known in the art. Methods for determining the ability of an agent to bind to HSC70 are known in the art.

In certain embodiments, the agent comprises a detectable tag. For example, the agent may comprise a F_{C} region of an antibody and/or a part thereof.

In certain embodiments, the detectable tag is an exogenous tag, such as the Fc region of an antibody. In certain embodiments, the detectable tag comprises a Fc region of an antibody and/or a part thereof.

In certain embodiments, the detectable tag comprises all or part of the agent, such as an antigenic region of the polypeptide that can be recognised by an antibody.

In certain embodiments, the detectable tag is associated with a detectable agent or is a part of the detectable agent. For example the detectable agent may include the detectable tag as a fusion partner, a labelled amino acid or labelled nucleotide. Examples of suitable detectable tags include antigens, enzymes, fluorophores, quenchers, radioactive isotopes and luminescent compounds or labels. The detectable tag may be detected directly or indirectly via a further molecule that can produce a detectable signal. In certain embodiments, the detectable agent comprises an antigen. Examples of antigens that may be used as a detectable tag include suitable antigenic components of the detectable agent that may be targeted by a secondary detectable agent. For example, a secondary antibody may also be used to detect an antigen on a detectable agent. The secondary antibody may, for example, be fluorescently or enzymatically labelled.

In certain embodiments, the detectable tag comprises a fluorophore. Examples of fluorophores that may be used as detectable tags include, for example, resorufin, fluorescein (fluorescein isothiocyanate, FITC), rhodamine (tetramethyl rhodamine isothiocyanate, TRITC), green fluorescent protein (GFP), phycobiliproteins (allophycocyanin, phycocyanin, phycoerythrin and phycoerythrocyanin, lanthanide ions such as one or more of Eu3+, Sm3+, Tb3+, and Dy3+ or complexes thereof, suitable derivatives of the foregoing or combinations thereof. In certain embodiments, detectable tag may be part of the one or more different detectable agents (e.g. in the form of a fusion protein or a protein comprising fluorescent amino acids).

In certain embodiments, the detectable tag comprises a luminescent compound or label. Luminescent compounds or labels that may be used as detectable tags include, for example, chemiluminescent and/or bioluminescent compounds. These compounds may be used to label a detectable agent. The presence of a chemiluminescent-tag may be determined by detecting the presence of luminescence that arises during the course of a chemical reaction. Examples of useful chemiluminescent labelling compounds are luminol, isoluminol, theromatic acridinium ester, imidazole, acridinium salt and oxalate ester or combinations thereof. Bioluminescence is a type of chemiluminescence found in biological systems in which a catalytic protein increases the efficiency of the chemiluminescent reaction. The presence of a bioluminescent antibody is determined by detecting the presence of luminescence. Examples of bioluminescent compounds include luciferin, luciferase and aequorin.

In certain embodiments, detectable tag comprises one or more lanthanide ions. In certain embodiments, the lanthanide ion may be Eu³⁺, Sm³⁺, Tbh³⁺, and Dy³⁺. In certain embodiments, one of the detectable tags comprises Eu³⁺ and another of the detectable tags comprises Sm³⁺.

In certain embodiments, the detectable tag comprises one or more enzymes that convert a substrate into a detectable product. Enzymes that may be used as detectable tags include, for example, enzymes that result in the conversion of a substrate into a detectable product (generally resulting in a change in colour or fluorescence or generation of an electrochemical signal). Such enzymes may include, for example, horseradish peroxidase (HRP), alkaline phosphatase (AP), β-galactosidase, acetylcholinesterase, luciferase, catalase or combinations thereof. Depending on the enzyme and substrate used, detection may be performed with a spectrophotometer, fluorometer, luminometer, and/or other electrochemical detection means. In certain embodiments, the enzyme may be horse radish peroxidase, alkaline phosphatase, and beta-galactosidase. Other enzymes may also be used.

In certain embodiments, the detectable tag comprises a radioactive isotope, Radioactive isotopes that may be used as detectable tags include, for example, ³H, ¹⁴C, ³²P, ³⁵S, or ¹³¹I. The radioisotope may be conjugated to a detectable agent or incorporated into a detectable agent by translation of mRNA encoding the detectable agent in the presence of radiolabelled amino acids. Radioisotopes and methods for conjugating radioactive isotopes to molecules such as proteins are known. Radioisotopes may be detected using gamma, beta or scintillation counters.

Certain embodiments of the present disclosure provide a method of enriching for immature, uncommitted stromal stem cells from a population of cells comprising stromal stem cells, the method comprising using an agent as described herein and/or a polypeptide as described herein to enrich the stromal stem cells from the population of cells.

Certain embodiments of the present disclosure provide a method of isolating immature, uncommitted stromal stem cells from a population of cells comprising stromal stem cells, the method comprising using an agent as described herein and/or a polypeptide as described herein to isolate the stromal stem cells from the population of cells.

Certain embodiments of the present disclosure provide a nucleic acid encoding a polypeptide as described herein, and/or a vector comprising a nucleic acid encoding a polypeptide as described herein.

The term “nucleic acid” as used herein refers to an oligonucleotide or a polynucleotide and includes for example DNA, RNA, DNA/RNA, a variant or DNA and/or RNA (for example a variant of the sugar-phosphate backbone and/or a variant of one or more bases, such as methylation), and may be single stranded, double stranded, non-methylated, methylated or other forms thereof. In certain embodiments, the nucleic acid is a non-naturally occurring nucleic acid, a naturally occurring nucleic acid, a nucleic acid of genomic origin, a mitochondrial nucleic acid, a nucleic acid of cDNA origin (derived from a mRNA), a nucleic acid derived from a virus, a nucleic acid of synthetic origin, a single stranded DNA, a double stranded DNA, an analogue of DNA and/or RNA, and/or a derivative, fragment and/or combination of any of the aforementioned. Examples of derivatives also include nucleic acids that have a blocking group at the 5′ and/or 3′ ends for example to improve stability, and/or nucleic acids fused to other molecules. Other types of nucleic acids are contemplated. Methods for producing nucleic acids are known and include for example nucleic acids produced by recombinant DNA technology or nucleic acids produced by chemical synthesis.

The term “nucleic acid” as used herein also refers to a specified nucleic acid, or a nucleic acid comprising a nucleotide sequence which is the complement of the nucleic acid, a nucleic acid comprising a nucleotide sequence with greater than 70%, 75%, 800%, 85%, 90%, or 95% sequence identity to the specified nucleic acid, or a nucleic acid comprising a nucleotide sequence with greater than 70%, 75%, 80%, 85%, 90% or 95% sequence identity to the complement of the specified nucleic acid. Other levels of sequence identity are contemplated.

Certain embodiments of the present disclosure provide a host cell, such as a prokaryote cell or a eukaryotic cell comprising a vector as described herein.

Certain embodiments of the present disclosure provide a kit, or a combination product, for performing a method as described herein.

A kit for use in a method as described herein may, for example, contain one or more of the following: a HSC70 binding agent, such as an anti-HSC70 antibody, and which may for example be biotinylated or fluorescently labelled; a STRO-1 antibody, and which may for example be biotinylated or fluorescently labelled; a HSP70 binding agent, such as an anti-HSP70 antibody, and which may for example be biotinylated or fluorescently labelled; one or more secondary reagents, such as antibodies, and which may also be biotinylated or fluorescently labelled; buffers; media; standards; controls; reagents and instructions/methodology; and one or more reagents as described in the Examples.

Certain embodiments of the present disclosure provide a kit for enriching and/or isolating immature, uncommitted stromal stem cells, the kit comprising a HSC70 binding agent.

HSC70 binding agents are as described herein. In certain embodiments, the HSC70 binding agent comprises an antibody and/or antigen binding part thereof.

Certain embodiments of the present disclosure comprise a kit, or a combination product, comprising a HSC70 binding agent and a HSP70 binding agent.

Certain embodiments of the present disclosure comprise a kit, or a combination product, comprising a STRO-1 antibody and a HSP70 binding agent.

Standard techniques may be used for recombinant DNA technology, oligonucleotide synthesis, antibody production, peptide synthesis, tissue culture and transfection. Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See for example, Green M R and Sambrook J, Molecular Cloning: A Laboratory Manual (4th edition), Cold Spring Harbor Laboratory Press, 2012, herein incorporated by reference.

Certain exemplary embodiments are illustrated by some of the following examples. It is to be understood that the following description is for the purpose of describing particular embodiments only and is not intended to be limiting with respect to the above description.

### Example 1—the STRO-1 Antigen is Localised to Cholesterol Rich Micro-Domains

Flow cytometry: UE7T-13 cells were harvested by trypsinisation and resuspended in blocking buffer (human serum (5%), bovine serum albumin (BSA) (1%), fetal calf serum (FCS) (5%), penicillin 5000u/mL and streptomycin 5000u/mL) at 2×10⁷/mL for 30 minutes on ice. 1×10⁶ cells were then incubated with 50 L of 1A6.12 hybridoma supernatant (IgM isotype mouse monoclonal antibody (mAB) raised against Salmonella) or STRO-1 (IgM isotype) hybridoma supernatant for 1 hour on ice. Cells were pelleted by centrifugation, washed twice (wash buffer: Hanks buffered salt solution, FCS (5%)) then incubated with fluorescein labelled goat anti-mouse IgM secondary antibody for 30 minutes on ice. Cells were washed; fixed (0.1% formaldehyde, 0.02% azide, 110 mM glucose) and fluorescent signal detected using the BD FACSCanto II flow cytometer (BD Biosciences). Immunofluorescence staining: UE7T-13 cells were grown on glass 8 well chambers slides and cell staining was performed in situ as per flow cytometry. Cells labelled with STRO-1 were detected using confocal microscopy (LSM400, Zeiss, Oberkochen, Germany)). Cholesterol sequestration: UE7T-13 cells were incubated with methyl β-cyclodextrin (MβCD) for 1 hour at 37° C. then washed twice in wash buffer. Flow cytometry was performed as detailed above. Prior to fixation the cell viability dye 7-Aminoactinomycin D (7AAD) was incubated with the labelled cells for 30 minutes on ice.

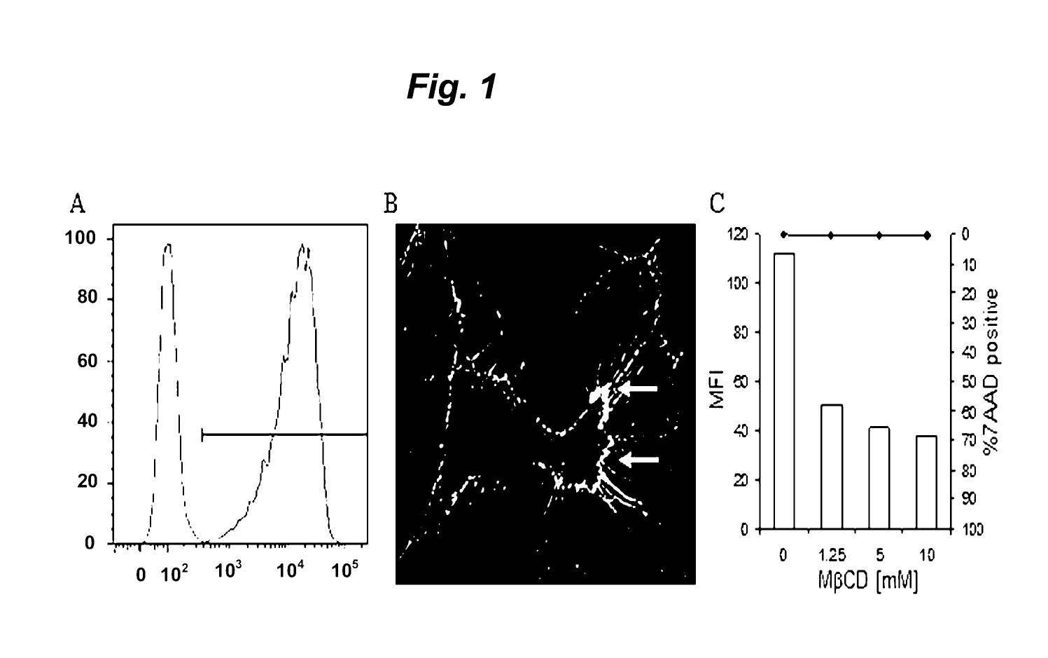

The data is shown FIG. 1. (A) UE7T-13, an immortalised human bone marrow derived mesenchymal stromal cell line, has abundant STRO-1 bindings sites on the cell surface as evidenced by the high mean fluorescence intensity (MFI) observed in STRO-1 labelled cells using flow cytometry (1A6.12 MFI=112; STRO-1 MFI=17188) (B) Confocal microscopy was used to detect STRO-1 binding to live UE7T-13 cells. Arrows indicate STRO-1 binding to cellular protrusions or pseudopodia in cultured UE7T-13 cells by immuno-fluorescence. (C) Sequestration of cholesterol from the plasma membrane using methyl β-cyclodextrin (MβCD) reduces STRO-1 binding to UE7T-13 cells suggesting the STRO-1 antigen is localised to cholesterol rich micro-domains on the cell surface. Treatment with MβCD did not reduce cell viability, as assessed using 7AAD staining, a fluorescent dye that is precluded from entering viable cells.

### Example 2—STRO-1 Binds to a 70 kDa Protein

Protein lysates were prepared from UE7T-13 cells using RIPA buffer. Equivalent amounts (50 μg) of protein were resolved by SDS-PAGE under non-reducing and reducing (5% 2-Mercaptoethanol) conditions. Resolved proteins were transferred electrophoretically to nylon membranes (192 mM glycine, 25 mM Tris, 15% methanol at 250 mA for 2 hours). Membranes were blocked using 2.5% (w/v) skim milk powder in Tris-buffered saline/0.1% Tween 20 (TBS-T) (blocking buffer) then incubated with STRO-1 or 1A6.12 hybridoma supernatant (dilute 1:3 with blocking buffer) or 10 g/mL purified STRO-1 in blocking buffer (R&D Systems; MAB1038) overnight at 4° C. Membranes were washed in TBS-T then incubated with an alkaline phosphatase-conjugated anti-IgM secondary antibody in blocking buffer. Bound secondary antibody was visualised using a Chemidoc Imager (BIO-RAD) in the presence of an enhanced chemi-fluorescence substrate.

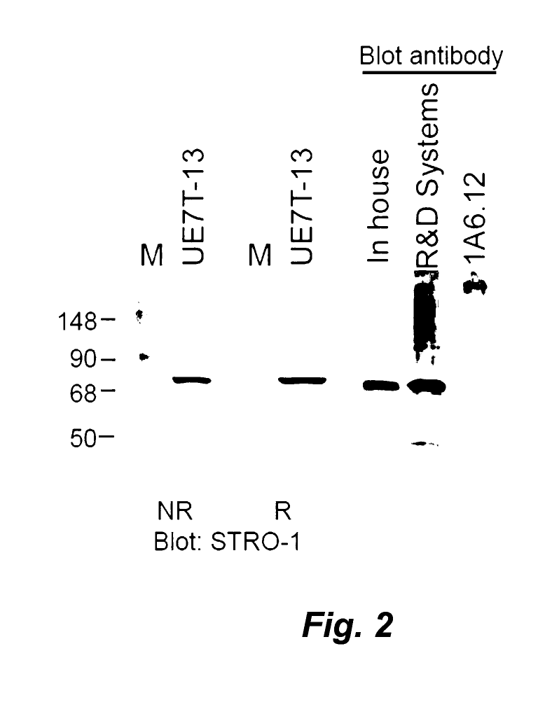

The data is shown in FIG. 2. STRO-1 binds to a 70 kDa protein. Protein lysates prepared from UE7T-13 cells under non-reducing (NR) and reducing (R) conditions were probed using STRO-1 hybridoma supernatant. STRO-1 bound to an approximately 70 kDa protein irrespective of protein reduction. To determine if the in-house STRO-1 hybridoma supernatant gave similar blotting patterns to STRO-1 sourced commercially, purified STRO-1 was purchased from R&D Systems (MAB1038) and a Western blot was performed. Both preparations bound a 70 kDa protein in UE7T-13 lysates. No protein was detected using an IgM control antibody 1A6.12 demonstrating that STRO-1 antibody binding was not due to non-specific binding of mouse IgM.

### Example 3—STRO-1 Binds to Heat Shock Cognate 70 (HSC70; HSPA8)

Two-dimensional gel electrophoresis and mass spectroscopy: Protein lysates from UE7T-13 cells were prepared using 2D buffer (7M Urea, 2M Thiourean and 4% CHAPS) and sonication. Proteins were then reduced and alkylated using 5 mM Tributyl phosphine (TBP) and 10 mM acrylamide respectively at room temperature (RT) for 90 minutes. Two-dimensional (2D) gel electrophoresis was performed in duplicate (first dimension isoelectric focusing pH 4-7 IPG strips followed by electrophoresis on 4-20% Bio-rad Criterion Gels). Resolved proteins were either electrophoretically transferred to a nylon membrane or immediately stained with SYPRO-RUBY, a non-specific protein stain. The nylon membrane was Western blotted using STRO-1 (as detailed in [00281]) then aligned with the SYPRO-RUBY stained duplicate. A region of the SYPRO-RUBY stained gel corresponding to the protein detected by Western blotting with STRO-1 was excised, digested with trypsin and peptides were extracted and desalted. Matrix assisted laser desorption ionisation mass spectroscopy (MALDI) was performed with an Applied Biosystems 4800 Plus MALDI TOF/TOF Analyser. From this analysis, the peptide peak lists were submitted to the database search program Mascot (Matrix Science Ltd, London, UK) and peptide sequences searched against Homo sapiens SwissProt database. Immunoprecipitations: UE7T-13 cell lysates prepare using RIPA buffer (1 mg) were incubated with anti-HSC70 mAB 1B5 (Enzo) or anti-HSP90 rabbit polyclonal antibody (Santo Cruz) overnight at 4° C. Immuno-precipitating antibody was captured using Protein G sepharose for 1 hr at 4° C. The Protein G sepharose and bound anti-body/antigen complex was pelleted by centrifugation (200×g) and washed 5 times in 0.5×RIPA buffer. The complex was heated (100° C. for 5 min) in reducing buffer and proteins resolved by SDS-PAGE. STRO-1 Western blots were performed as described in Example 2. siRNA: small interfering RNA (siRNA) that targets the HSPA8 transcript (Silencer Select HSPA8 siRNA, 4390824, Life technologies) and a negative control siRNA (Silencer Select Negative Control #1, 4390843) were introduced into UE7T-13 cells using Lipofectamine RNAiMAx (Life Technologies). After 3 days in culture, protein lysates were prepared and a STRO-1 Western blot was performed as described in Example 2.

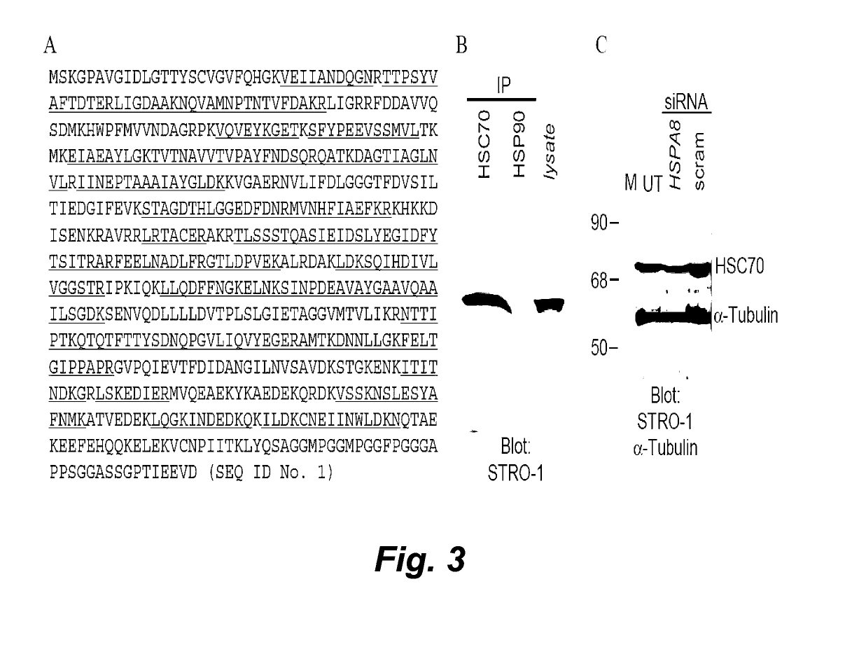

The data is shown in FIG. 3. (A) To identify the protein bound by STRO-1, 2-dimensional gel electrophoresis, coupled with Western blotting, was performed. Proteins bound by STRO-1 were picked and subjected to tandem mass spectroscopy. From this analysis, peptides with sequences identical to heat shock cognate 70 (HSC70, HSPA8) were sequenced. Sequenced peptides covering 600/% of HSC70 were identified (shown as underlined). (B) To confirm that STRO-1 binds to HSC70, HSC70 was immune-precipitated from UE7T-13 lysates with an anti-HSC70 antibody (1B5, STRESSGEN) and Western blotted with STRO-1. STRO-1 was found to bind to immuno-precipitated HSC70 but not HSP90. (C) To further confirm STRO-1 binds to HSC70, siRNA was used to inhibit translation of HSC70 RNA transcripts and the level of protein detected by STRO-1 was assessed by Western blotting. A significant reduction in blotted protein was observed in cells transfected with HSPA8 siRNA but not those transfected with scrambled siRNA. Taken together, these findings are consistent with STRO-1 binding to HSC70.

### Example 4—STRO-1 Binds to HSC70 and HSP70 and Cell Surface Binding of STRO-1 does not Correlate with HSC70 or HSP70 Protein Expression

Flow cytometry was performed as described in Example 1. Western blotting was performed as described in Example 2 on protein lysates obtained from multiple primary cells and cell lines using RIPA buffer. Recombinant human HSC70, HSP70 and GRP78 was sourced from ENZO.

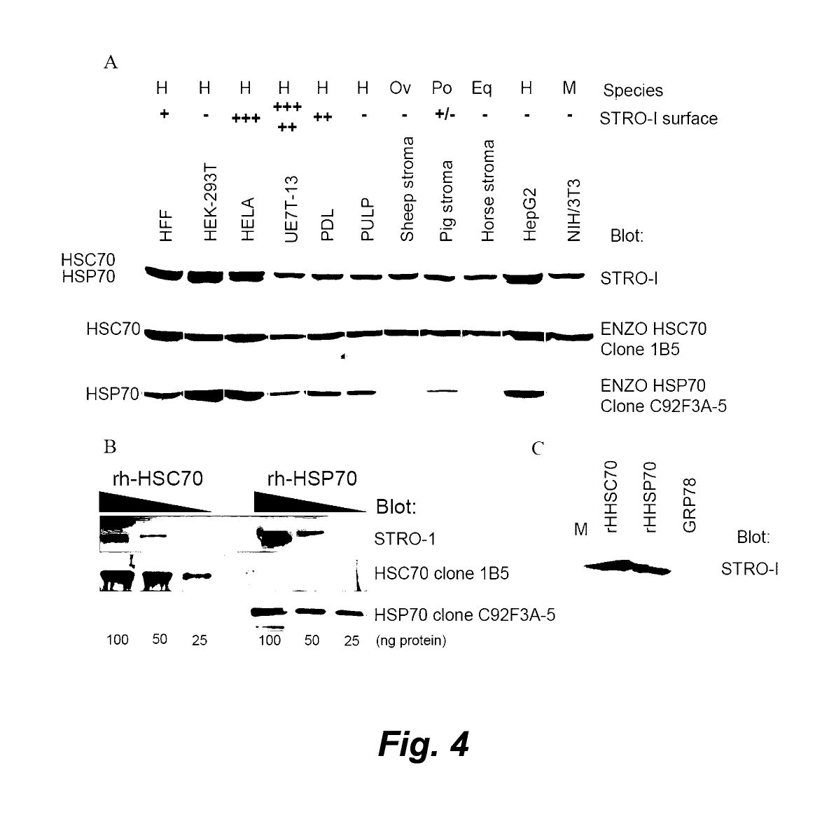

The data is shown in FIG. 4. (A) HSC70 is ubiquitously expressed and yet STRO-1 cell surface binding occurs in a limited number of cell types by flow cytometry. To determine if STRO-1 surface binding correlates with STRO-1 binding in cell lysates, flow cytometry and Western blotting was performed on a number of cell lines and primary cells from different species. Cell surface binding of STRO-1 is indicated as either negative (−) or positive (+). STRO-1 detects HSC70 in cellular lysates irrespective of cell surface binding. In some lysates such as HEK-293T, HELA and HepG2, STRO-1 detected 2 proteins of close molecular weight. (B) HSC70 is highly homologous to heat shock protein 70 (HSP70) suggesting STRO-1 might cross-react with both proteins. To test this possibility, Western blots were performed using purified human recombinant proteins. STRO-1 bound to both HSC70 and HSP70. (C) To test if STRO-1 binds to HSP70 family members non-specifically, STRO-1 binding to GRP78, another closely related family member was tested. STRO-1 bound HSC70 and HSP70 but not Grp78.

### Example 5—HSC70 is Present on the Surface of UE7T-13 Cells and Pre-Incubation of STRO-1 with Recombinant HSC70 Block STRO-1 Binding to UE7T-13 Cells

Cell surface biotinylation: UE7T-13 and HEK-293T cells were harvested by trypsinisation, washed in phosphate-buffered saline (PBS) then incubated with cell-impermeable EZ-Link Sulfo-NHS-LC-Biotin (0.5 mg/mL) for 1 hour on ice. The reaction was quenched upon addition of 100 mM glycine. Cell lysates were prepared using RIPA buffer. Immuno-precipitations: Biotinylated cell lysates (1 mg) were incubated with anti-HSC70 mAB 1B5 (Enzo) or purified Rat IgG overnight at 4° C. Immuno-precipitating antibody was then captured using Protein G sepharose for 1 hour at 4° C. The immuno-complex was pelleted by centrifugation (200×g) and washed 5 times in 0.5×RIPA buffer. The complex was heated (100° C. for 5 min) in reducing buffer and proteins resolved by SDS-PAGE. Biotinylated proteins were detected by Western blotting using Streptavidin-conjugated alkaline phosphatase. HSC70 protein was detected using STRO-1 by Western blotting. Protein blocking: Purified STRO-1 (R&D Systems) was incubated with and without recombinant human HSC70 (Enzo) in blocking buffer overnight at 4° C. A flow cytometry assay was then performed as detailed in Example 1.

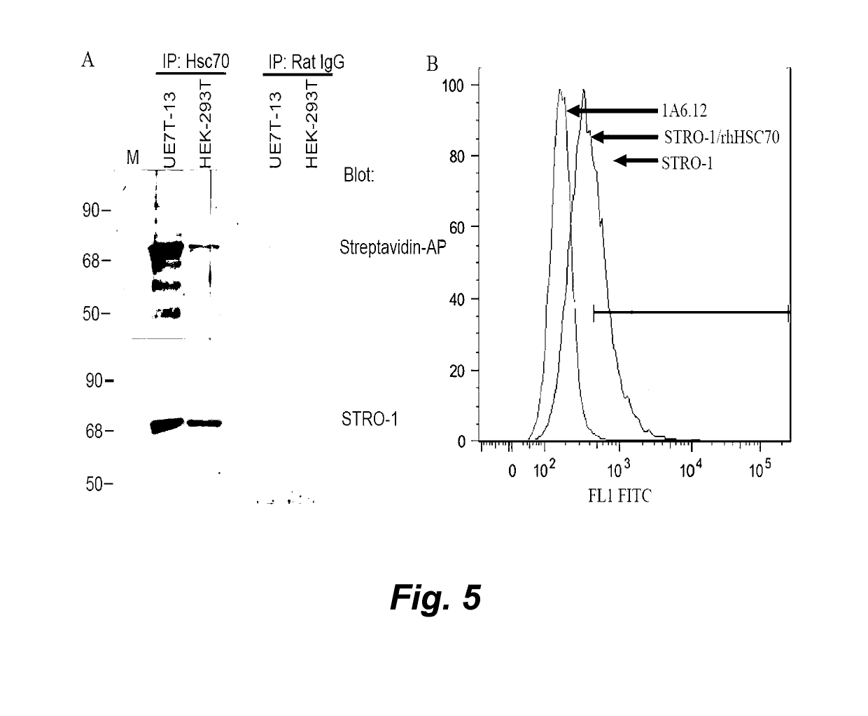

The data is shown in FIG. 5. (A) In order for STRO-1 to bind UE7T-13 cells, HSC70 must be exposed on the cell surface. To investigate this question, proteins present on the surface of UE7T-13 and HEK-293T cells (a cell line that does not bind STRO-1) were labelled with EZ-Link Sulfo-NHS-LC-LC-Biotin, a membrane impermeable biotinylation reagent. Cell lysates were prepared from labelled cells and HSC70 was immuno-precipitated using 1B5 (anti-HSC70 antibody, STESSGEN). Biotinylated HSC70 was then detected using Streptavidin-AP. HSC70 was biotinylated in UE7T-13 cells but not HEK293T cells consistent with cell surface binding of STRO-1 as detected by flow cytometry (FIG. 4). Blots were re-probed with STRO-1 to show that similar levels of HSC70 were immuno-precipitated from both cell lines. (B) To demonstrate more directly that STRO-1 binds to HSC70 on the surface of UE7T-13 cells, STRO-1 was pre-incubated with rh-HSC70 overnight at 4° C. The antibody/protein complex was then incubated with UE7T-13 cells and any STRO-1 that bound to the cell surface was detected using a fluorescently tagged secondary antibody and flow cytometry. Histograms showing the fluorescence signal resulting from the incubation of cells with 1A6.12 (IgM isotype control), STRO-1 alone and the STRO-1/rhHSC70 complex are overlaid. Pre-incubation of STRO-1 with rhHSC70 reduced STRO-1 binding to UE7T-13 cells (300/% reduction in MFI). Taken together, these data suggest that STRO-1 binds to HSC70 on the surface of UE7T-13 cells.

### Example 6—STRO-1 Binds to the ATPase Domain of HSC70

Genetic engineering truncations: truncated forms of HSPA8 were generated using the polymerase chain reaction with PfuTurbo (a high fidelity thermostable polymerase), sequenced and cloned into the retro-viral expression vector pRUFiG2-HA. pRUFiG2-HA has a N-terminal Hemagglutinin sequence (HA) inframe to the ATG of a NdeI restriction site. Truncated forms of HSPA8 were introduced into UE7T-13 cells using retro-viral transduction as previously described [Isenmann S et al., Stem Cells, 2009, 10:2457-68]. Immuno-precipitations using an anti-HA antibody (05-904; Millipore) was performed as described in Example 3.

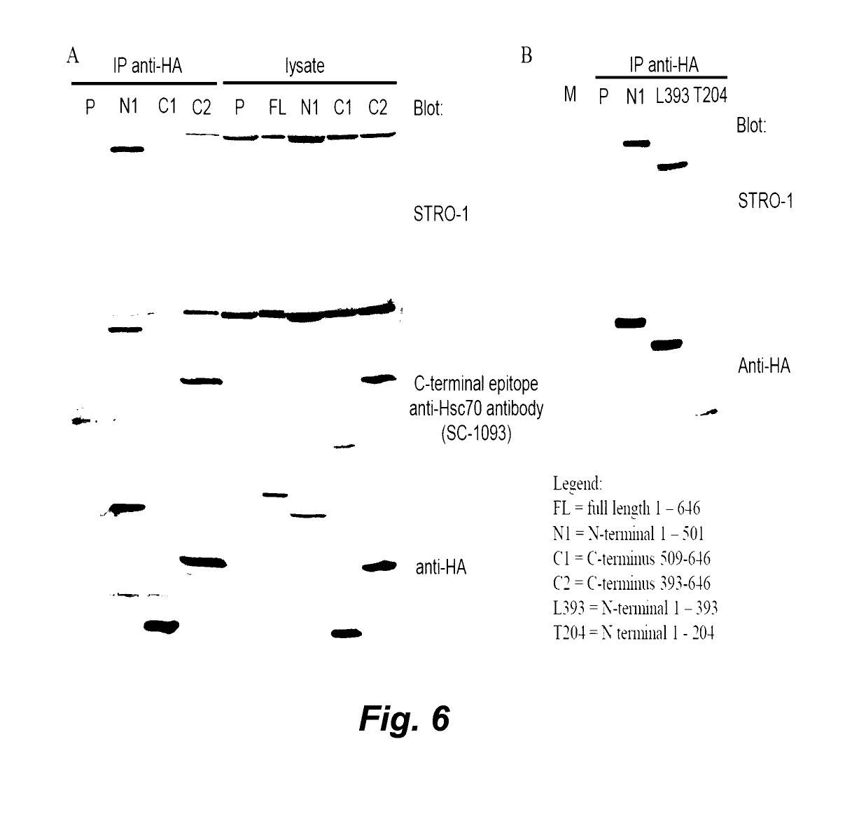

The data is shown in FIG. 6. STRO-1 binds to the ATPase domain of HSC70. To map the STRO-1 epitope of HSC70, a HA-tag was added at the N-terminus of human HSC70 and a series of C-terminal and N-terminal truncations were engineered. Truncations were introduced into UE7T-13 cells by retro-viral transduction. (A) N-terminally HA-tagged HSC70 truncations were immuno-precipitated and STRO-1 binding assessed by Western blotting (Upper panel). STRO-1 binds to the N-terminal (Ni, 1-501) fragment but not to the C-terminal domains. C-terminal truncations were detected using a C-terminal specific antibody (middle panel) and anti-HA (lower panel) demonstrating that the truncated proteins were expressed. (B) To investigate the epitope further, 2 additional truncations of the N-terminal ATPase domain were examined. STRO-1 bound to Ni and L393 (1-393) but not to T204 (1-204) (Upper panel). Protein expression was confirmed by blotting with anti-HA (Lower panel). Taken together, this data suggest STRO-1 binds to the ATPase domain of HSC70 in a region that spans amino acids 204-393.

### Example 7—Fine Mapping the STRO-1 Epitope on HSC70

Peptide arrays: A peptide array spanning the first 393 amino acids of human HSC70 was synthesized by INTAVIS Bioanalytical Instruments AG (Heidelberg, Germany). Duplicate arrays were blocked in 2.5% (w/v) skim milk for 2 hours, rinsed in TBS-T then probed with either 1A6.12 or STRO-1 supernatant (diluted 1:2 in skim milk) overnight at 4° C. Arrays were washed with TBS-T then incubated with an alkaline phosphatase-conjugated anti-IgM secondary antibody at room temperature for 1 hr. After washing with TBS-T, bound secondary antibody was visualised using a Typhoon FLA 7000 imager (GE HealthCare Life Sciences) in the presence of an enhanced chemi-fluorescence substrate. Deletion mutant analysis: A deletion mutant of HSC70 in which the first 4 amino acids of the putative STRO-1 epitope was deleted (AISEN) was created using QuickChange Site-Directed mutagenesis. A stable cell line variant of UE7T-13 expressing AISEN was created (L393 AISEN) using retro-viral transduction. Protein lysates were prepared from L393 control and L393 AISEN UE7T-13 variants using RIPA buffer. Immune-precipitations (1 mg) were then performed using an anti-HA antibody or an isotype matched mouse immunoglobulin as described in Example 3. Immune-precipitated proteins were resolved by SDS-PAGE, transferred to nylon membranes then probed using STRO-1 hybridoma supernatant as described in Example 2. After visualisation of STRO-1 antibody binding, membranes were stripped (Alpha Diagnostics), blocked and re-probed.

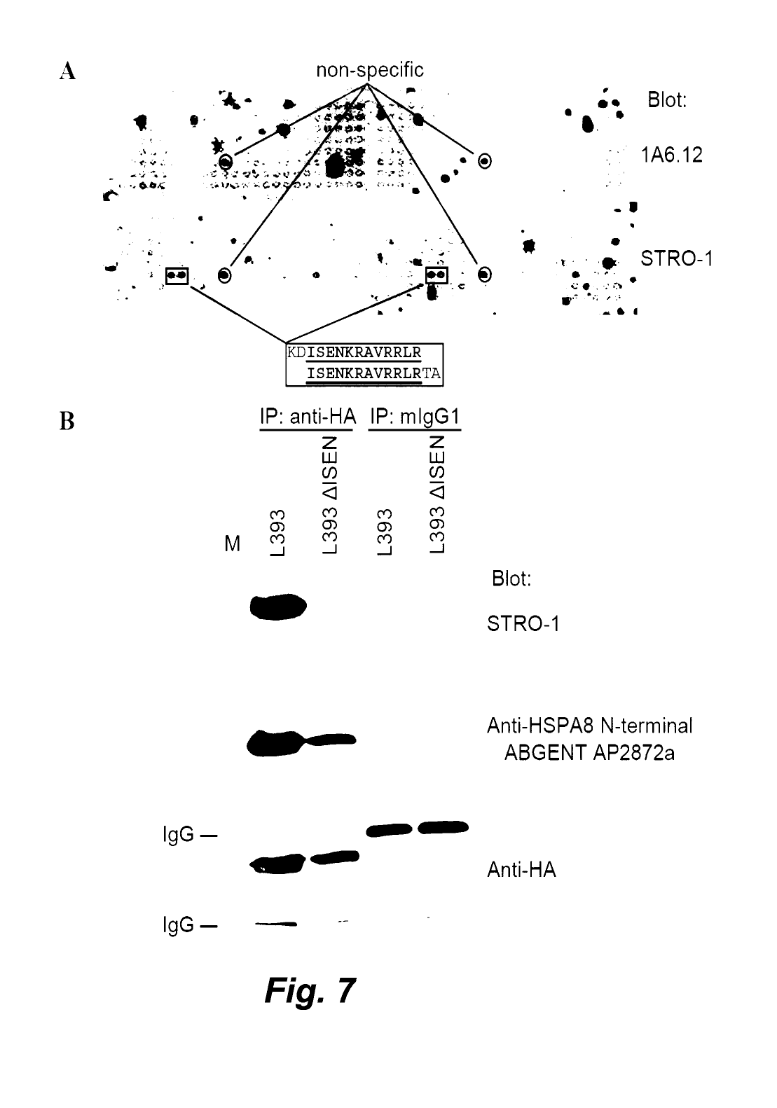

The data is shown in FIG. 7. (A) To fine map the STRO-1 epitope on HSC70, an array of peptides spanning the first 393 amino acids of human HSC70 was synthesized. Each peptide is 14aa in length and is off-set by 2aa resulting in an array of 192 peptides spotted in duplicate onto a glass slide. Peptide arrays were probed with 1A6.12, IgM isotype control or STRO-1. STRO-1, and not 1A6.12, bound two consecutive peptides on duplicate spots on the array (black boxes). These peptides corresponded to amino acids 251-266 and lie within the region (204-393) identified by the deletion mapping (FIG. 6) (B). To verify the array data, a deletion mutant, in which the first 4aa of the putative STRO-1 epitope was deleted (L393-AISEN), was introduced into UE7T-13 cells. Anti-HA immuno-precipitations using lysates prepared from control L393 and L393-AISEN expressing cells were performed. STRO-1 bound L393, however, no binding to L393-AISEN was detected. In contrast, a HSC70 antibody raised to a peptide corresponding to aa 82-110 (ABGENT, AP2872a) bound both L393 and L393-AISEN. To confirm loading differences, membranes were stripped and re-probed with anti-HA. These data suggest STRO-1 binds to an epitope in the ATPase domain of HSC70 in the region corresponding to amino acids 251-266.

STRO-1 bound to consecutive peptides suggesting the epitope is confined to a 12 aa consensus sequence. STRO-1 also bound to duplicate peptides on the array demonstrating reproducibility. The IgM isotype control antibody 1A6.12 did not bind to the STRO-1 epitope demonstrating specificity. The peptides bound by STRO-1 maps to the region identified using the deletion mutants i.e. within T204-F293 further supporting the array experiments. Such peptides have a variety of possible uses, such as blocking STRO-1 binding.

The amino acid sequences of the peptides bound by STRO-1 were as follows:

(SEQ ID NO. 2)
KDISENKRAVRRIR
(SEQ ID NO. 3)
ISENKRAVRRLARTA

Accordingly, the epitope bound by STRO-1 is as follows:

(SEQ ID NO: 4)
ISENKRAVRRLAR.

### Example 8—Recombinant HSC70 Blocks STRO-1 Binding to the Cell Surface

A construct was generated using standard techniques in which the first 393 amino acids of human HSC70 was cloned in-frame with Glutathione S-transferase to make a N-terminal GST fusion protein (GST-L393). The Glutathione-S-transferase (GST) gene fusion system is described, for example, in Current Protocols in Protein Science. 2008 May; Chapter 6: Unit 6.6. Expression and purification of GST fusion proteins, Harper S1, Speicher D W, doi: 10.1002/0471140864.

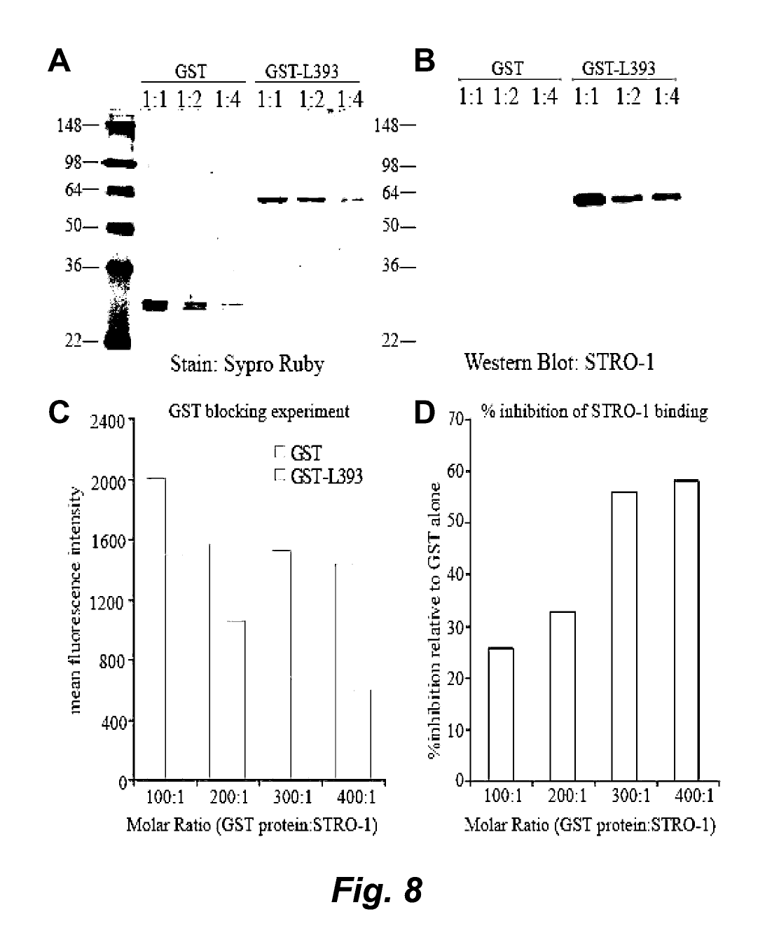

Recombinant GST and GST-L393 were purified from bacteria lysates using Glutathione sepharose affinity chromatography. Purified proteins were dialysed against PBS and quantitated. Serial dilutions of purified proteins were resolved by SDS-PAGE and purity checked using a non-specific protein stain, as shown in FIG. 8A.

To ensure the STRO-1 epitope was maintained in the GST-L393 fusion protein, a Western blot using STRO-1 was performed, as shown in FIG. 8B. To determine if STRO-1 binds to HSC70 on the cells surface, a competition binding assay was performed. Purified GST or GST-L393 was incubated with STRO-1 at different molar ratios in PBS overnight at 4° C. Complexes were then incubated with UE7T-13 cells for 45 minutes on ice. Cells were washed 2× in wash buffer then incubated with 1:100 anti-IgM-PE conjugate for 45 minutes on ice. Cells were washed 2× and STRO-1 binding was detected using flow cytometry. Pre-incubation of STRO-1 with GST-L393, but not GST alone, caused a dose-dependent decrease in STRO-1 binding to the cell surface suggesting STRO-1 binds to HSC70 on the cell surface (FIG. 8C).

The level of antibody blocking caused by pre-incubation with GST-L393 was determined relative to the fluorescence signals recorded for pre-incubation with GST alone. Pre-incubation with GST-L393 caused a 600/% reduction in antibody binding (FIG. 8D).

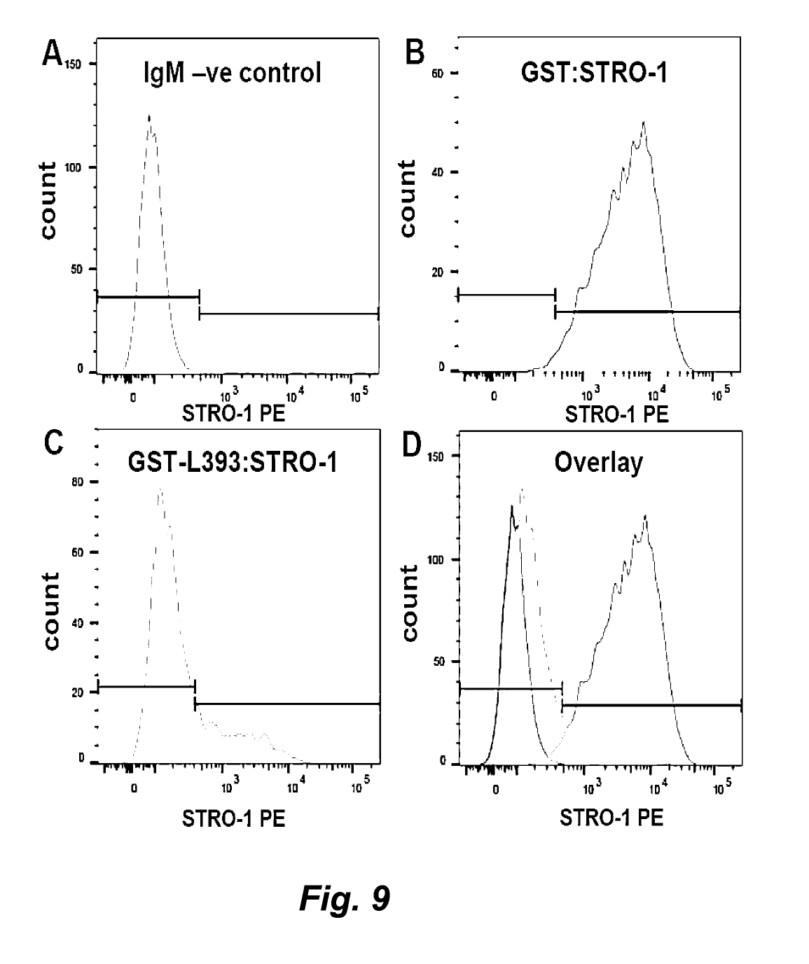

Flow cytometry histograms showing fluorescence signal generated following the incubation of UE7T-13 cells with an anti-Salmonella IgM negative control (1A6.12) (FIG. 9A), STRO-1 pre-incubated with GST 300:1 (FIG. 9B), STRO-1 pre-incubated with GST-L393 300:1 (FIG. 9C) and an overlay of A-C(FIG. 9D).

### Example 10—the STRO-1+Ve/HSP70 −Ve Fraction of Human BMMNCs Contains all CFU-F Activity

Our prediction was that the STRO-1 +ve cell fraction within the bone marrow contains all the immature stromal cells that give rise to fibroblastic colonies (colony forming unit fibroblastic CFU-F). Given that STRO-1 binds to both HSC70 and HSP70, the immuno-phenotype of the CFU-F cells is unclear.

We therefore sought to purify the STRO-1 +ve fraction of cells from human bone marrow mononuclear cells (BMMNCs), sub-divide the STRO-1 +ve fraction based on HSP70 binding, sort and isolate the populations and assay for CFU-F activity.

Bone marrow mononuclear cells (BMMNCs) were obtained from iliac crest bone marrow aspirates from haematologically normal donors. BMMNCs were pelleted by centrifugation and resuspended in blocking buffer as detailed above. STRO-1 hybridoma supernatant was added and cells incubated on ice for 1 hour. Cells were then washed as detailed in Example 4 and resuspended in MACs buffer (PBS, 2% FCS, 5 mM EDTA). Anti-IgM microbeads (Miltenyi) were added to the cells and incubated on ice for 30 minutes. Beads and cell were washed then loaded onto a magnetic activated cell separation (MACs) column (MS separation column, Miltenyi) mounted on a magnetic stand. The column was washed with wash buffer, removed from the magnetic stand and cells that bound to the column were eluted. STRO-1+ cells were then incubated with Phycoerythrin labelled anti-IgM antibody and fluorescein labelled cmHSP70.1 (anti-HSP70) for one hour on ice. Cell were washed and discreet cell populations isolated by fluorescent activated cells sorting (BD FACSAria Fusion, BD Biosciences). Sorted cells were counted then seeded in 6 wells plates at 1×10⁵ and 3×10⁵ cells per well in growth media supplemented with 20% FCS. After 14 days, colonies were stained with toluidine blue (0.1% toluidine blue in 1% paraformaldehyde) and enumerated.

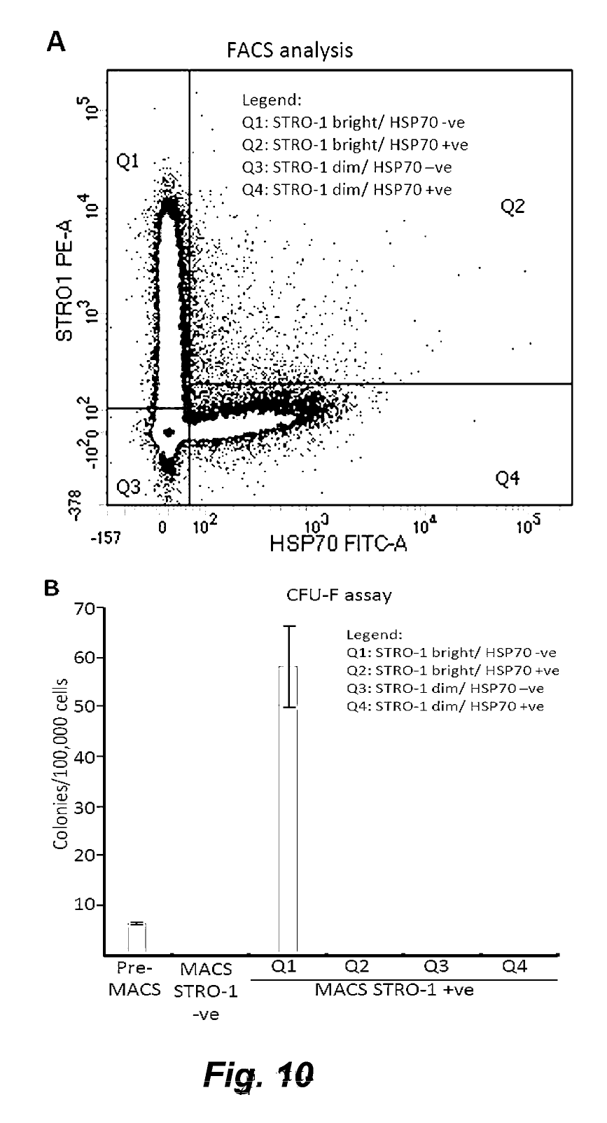

The data is shown in FIG. 10, which shows that the STRO-1 +ve/HSP70-ve fraction of human BMMNCs contains all CFU-F activity.

BMMNCs isolated from the iliac crest were incubated with STRO-1 and STRO-1 +ve cells (MACS STRO-1 +ve) were purified from the STRO-1 −ve cells (MACS STRO-1 −ve) by magnetic activated cell separation (MACs) using Miltenyi microbeads. (A) MACS STRO-1 +ve cells were then incubated with anti-IgM-PE (to detected STRO-1 bound to the cells) and anti-HSP70-FITC (cmHSP70.1). The cell population was then analysed by flow cytometry and cell populations (Q1-Q4 as shown in FIG. 10. A) were purified using fluorescence activated cell sorting (FACs). (B) Purified cells were then counted and plated. The number of colony forming units fibroblastic (CFU-F) were counted and normalised to the number of cells plated.

Conclusion: The cell population isolated using STRO-1 includes cells that also express HSP70 on the cell surface. However, all CFU-F activity is limited to the STRO-1 bright/HSP70 −ve population.

Based on the mean fluorescence intensity of pre-MACS versus STRO-1 +ve cells, the enrichment of cells was found to be greater than 12 fold.

### Example 11-20-202s and STRO-1 have Different Antigenic Specificities

Ab 20-202s is a monoclonal antibody that was generated using the human embryonic stem cell line (Miz-hES1) as an immunogen in mice (Sun Y S et al., Stem Cells 2005; 23: 1502-1513). It was found that Ab 20-202s bound to the surface of Miz-hES1 cells and, using immuno-precipitation and mass spectroscopy, HSC70 was identified as the target of 20-20s.

We therefore decided to obtain Ab 20-202s and compare the binding pattern of Ab 20-20s and STRO-1 using flow cytometry and Western blotting.

Flow cytometry was performed as described in Example 4 using purified 20-202s at 20 g/mL. Bound 20-202s was detected using a fluorescein labelled anti-mouse IgG antibody. Flow assays were analysed using the BD FACSCanto II flow cytometer (BD Biosciences). Purified recombinant proteins and protein lysate from cell lines were resolved by SDS-PAGE and transferred to nylon membranes as described herein. Western blots using 20-202s and STRO-1 were performed as described herein with the exception that bound 20-202s was detected using goat anti-mouse Alkaline phosphatase conjugated secondary antibody.

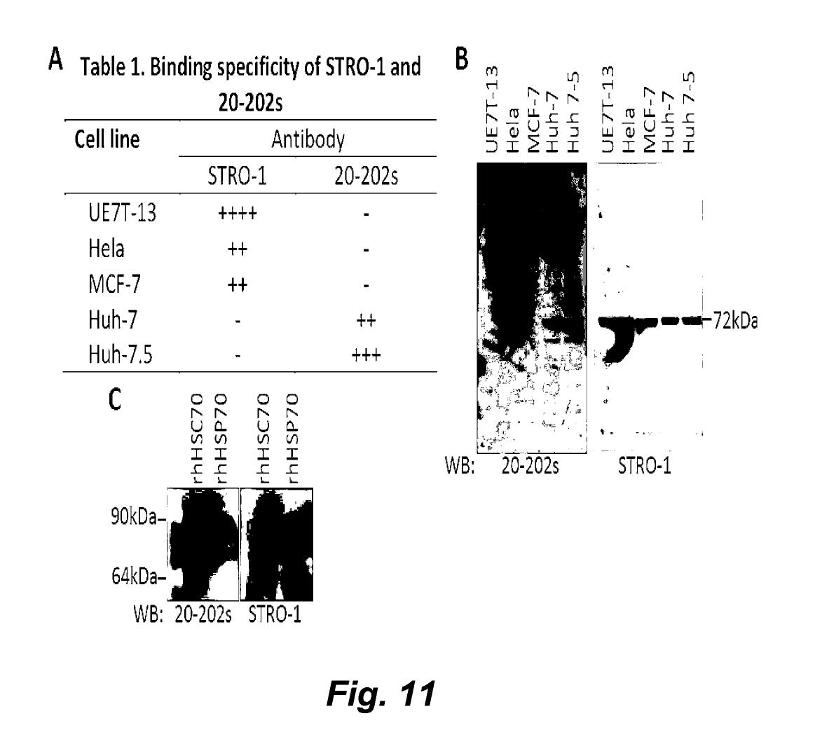

The data is shown in FIG. 11. (A) flow cytometry was performed to test the binding of Ab 20-202s and STRO-1 using cell lines previously shown to bind STRO-1. It was found that there is no overlap in antibody binding to the cell lines suggesting Ab 20-202s and STRO-1 bind to different epitopes on HSC70 that are cell line dependent. (B) By Western blot, Ab 20-202s binds to a 72 kDa protein but only in those cell lines where surface binding was shown by flow cytometry. In contrast, STRO-1 binds to HSC70 and HSP70 in all cell lines. (C) To test the specificity of Ab 20-202s, Western blots were performed using purified recombinant HSC70 and HSP70. Ab 20-202s does not bind (under reduced or non-reduced conditions (data not shown)) to recombinant HSC70 or HSP70.

These studies demonstrate that although Sun et al (2005) indicated that Ab 20-202s binds to HSC70 on Miz-hES1 cells, in our hands the antibody does not bind to HSC70, and accordingly the presence of HSC70 on Miz-hES1 cells is uncertain.

We conclude that either Ab 20-202s does not bind to HSC70 or that Ab 20-202s binds to a unique epitope on HSC70 that is not preserved in recombinant versions of this protein.

As used herein, the singular forms “a,” “an,” and “the” may refer to plural articles unless specifically stated otherwise.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

All methods described herein can be performed in any suitable order unless indicated otherwise herein or clearly contradicted by context. The use of any and all examples, or exemplary language (e.g., “such as”) provided herein, is intended merely to better illuminate the example embodiments and does not pose a limitation on the scope of the claimed invention unless otherwise claimed. No language in the specification should be construed as indicating any non-claimed element as essential.

The description provided herein is in relation to several embodiments which may share common characteristics and features. It is to be understood that one or more features of one embodiment may be combinable with one or more features of the other embodiments. In addition, a single feature or combination of features of the embodiments may constitute additional embodiments.

The subject headings used herein are included only for the ease of reference of the reader and should not be used to limit the subject matter found throughout the disclosure or the claims. The subject headings should not be used in construing the scope of the claims or the claim limitations.

Although the present disclosure has been described with reference to particular examples, it will be appreciated by those skilled in the art that the disclosure may be embodied in many other forms.

Future patent applications may be filed on the basis of the present application, for example by claiming priority from the present application, by claiming a divisional status and/or by claiming a continuation status. It is to be understood that the following claims are provided by way of example only, and are not intended to limit the scope of what may be claimed in any such future application. Nor should the claims be considered to limit the understanding of (or exclude other understandings of) the present disclosure. Features may be added to or omitted from the example claims at a later date.

1-60. (canceled)

61. A method of enriching for immature, uncommitted stromal stem cells from a population of cells comprising stromal stem cells, the method comprising enriching stromal stem cells from the population of cells using a HSC70 binding agent, and thereby enriching for immature, uncommitted stromal cells from the population of cells.

62. The method according to claim 1, wherein the immature, uncommitted stromal stem cells comprise immature, uncommitted mesenchymal stem cells.

63. The method according to claim 1, wherein the population of cells is obtained from placenta, umbilical cord, umbilical cord blood, tooth bud tissue, dentine/pulp tissue, periodontal ligament, gingival, skin, hair, follicle, amniotic fluid, adipose tissue, smooth muscle, skeletal muscle, tendon, ligament, bone, cartilage, bone marrow and/or peripheral blood.

64. The method according to claim 1, wherein the population of cells comprises stromal stem cells arising from pluripotent stem cells, induced pluripotent stem cells, cells arising from somatic nuclear transfer, and/or adult stem cells.

65. The method according to claim 1, wherein the method comprises binding of the HSC70 binding agent to cell surface HSC70.

66. The method according to claim 5, wherein the HSC70 binding agent does not substantially bind to cell surface HSP70.

67. The method according to claim 1, wherein the HSC70 binding agent comprises an antibody and/or an antigen binding part thereof.

68. The method according to claim 1, wherein the HSC70 binding agent comprises a HSC70 ligand.

69. The method according to claim 1, wherein the method comprises enriching for cells that express HSC70 as a cell surface marker.

70. The method according to claim 9, wherein the method comprises enriching for cells that express HSC70 as a cell surface marker and do not substantially express HSP70 as a cell surface marker.

71. The method according to claim 1, wherein the method comprises one or more of flow cytometry, magnetic activated cell sorting and antibody panning.

72. The method according to claim 11, wherein the method comprises enriching for cells that are HSC70⁺ and HSP70⁻.

73. The method according to claim 11, wherein the method comprises enriching for cells that are HSC70^{bright} and HSP70^{dim}.

74. The method according to claim 1, wherein the method is used to isolate immature, uncommitted stromal cells.

75. Cells enriched by the method according to claim 1.

76. A method of isolating immature, uncommitted stromal stem cells from a population of cells comprising stromal stem cells, the method comprising isolating stromal stem cells from the population of cells using a HSC70 binding agent.

77. Cells isolated by the method according to claim 16.

78. A method of identifying an agent for enriching, isolating and/or identifying an immature, uncommitted stromal stem cell, the method comprising:
determining whether a candidate agent binds to HSC70 on a stromal stem cell; and
identifying the candidate agent as an agent for enriching, isolating and/or identifying an immature, uncommitted stromal stem cells.

79. The method according to claim 18, wherein the method further comprises determining whether the candidate agent does not substantially bind to HSP70 on the stromal cell.

80. A kit for performing the method according to claim 1.


SEQUENCE LISTING (5 sequences)

SEQ ID NO: 1 - protein, 646 aa, Homo sapiens
MSKGPAVGIDLGTTYSCVGVFQHGKVEIIANDQGNRTTPSYVAFTDTERLIGDAAKNQVAMNPTNTVFDAKRLIGRRFDDAVVQSDMKHWPFMVVNDAGRPKVQVEYKGETKSFYPEEVSSMVLTKMKEIAEAYLGKTVTNAVVTVPAYFNDSQRQATKDAGTIAGLNVLRIINEPTAAAIAYGLDKKVGAERNVLIFDLGGGTFDVSILTIEDGIFEVKSTAGDTHLGGEDFDNRMVNHFIAEFKRKHKKDISENKRAVRRLRTACERAKRTLSSSTQASIEIDSLYEGIDFYTSITRARFEELNADLFRGTLDPVEKALRDAKLDKSQIHDIVLVGGSTRIPKIQKLLQDFFNGKELNKSINPDEAVAYGAAVQAAILSGDKSENVQDLLLLDVTPLSLGIETAGGVMTVLIKRNTTIPTKQTQTFTTYSDNQPGVLIQVYEGERAMTKDNNLLGKFELTGIPPAPRGVPQIEVTFDIDANGILNVSAVDKSTGKENKITITNDKGRLSKEDIERMVQEAEKYKAEDEKQRDKVSSKNSLESYAFNMKATVEDEKLQGKINDEDKQKILDKCNEIINWLDKNQTAEKEEFEHQQKELEKVCNPIITKLYQSAGGMPGGMPGGFPGGGAPPSGGASSGPTIEEVD

SEQ ID NO: 2 - protein, 14 aa, Homo sapiens
KDISENKRAVRRLR

SEQ ID NO: 3 - protein, 15 aa, Homo sapiens
ISENKRAVRRLARTA

SEQ ID NO: 4 - protein, 13 aa, Homo sapiens
ISENKRAVRRLAR

SEQ ID NO: 5 - protein, 574 aa, Homo sapiens
MGALRPTLLPPSLPLLLLLMLGMGCWAREVLVPEGPLYRVAGTAVSISCNVTGYEGPAQQNFEWFLYRPEAPDTALGIVSTKDTQFSYAVFKSRVVAGEVQVQRLQGDAVVLKIARLQAQDAGIYECHTPSTDTRYLGSYSGKVELRVLPDVLQVSAAPPGPRGRQAPTSPPRMTVHEGQELALGCLARTSTQKHTHLAVSFGRSVPEAPVGRSTLQEVVGIRSDLAVEAGAPYAERLAAGELRLGKEGTDRYRMVVGGAQAGDAGTYHCTAAEWIQDPDGSWAQIAEKRAVLAHVDVQTLSSQLAVTVGPGERRIGPGEPLELLCNVSGALPPAGRHAAYSVGWEMAPAGAPGPGRLVAQLDTEGVGSLGPGYEGRHIAMEKVASRTYRLRLEAARPGDAGTYRCLAKAYVRGSGTRLREAASARSRPLPVHVREEGVVLEAVAWLAGGTVYRGETASLLCNISVRGGPPGLRLAASWWVERPEDGELSSVPAQLVGGVGQDGVAELGVRPGGGPVSVELVGPRSHRLRLHSLGPEDEGVYHCAPSAWVQHADYSWYQAGSARSGPVTVYPYM